(19) United States
(12) Patent Application Publication
(10) Pub. No.: US 2024/0352412 A1
(43) Pub. Date: Oct. 24, 2024

(54) METHODS AND SYSTEMS FOR EXTENDED CULTURE OF HUMAN BLASTOIDS TO POST-IMPLANTATION STAGES ON 3D MATRICES

(71) Applicants: Thorold Theunissen, St. Louis, MO (US); Rowan Karvas, St. Louis, MO (US)

(72) Inventors: Thorold Theunissen, St. Louis, MO (US); Rowan Karvas, St. Louis, MO (US)

(73) Assignee: Washington University, St. Louis, MO (US)

(21) Appl. No.: 18/600,769

(22) Filed: Mar. 10, 2024

Related U.S. Application Data
(60) Provisional application No. 63/489,628, filed on Mar. 10, 2023.

(51) Int. Cl. C12N 5/073

(52) U.S. Cl. CPC C12N 5/0604; C12N 2500/44; C12N 2501/11; C12N 2501/235; C12N 2501/999; C12N 2509/00; C12N 2513/00; C12N 2533/90

(57) ABSTRACT

Methods for generating blastoids from naïve hPSCs are provided. Exemplary methods include exposing the naïve hPSCs to 5i/L/A, aggregating the exposed hPSCs in N2B27, and treating the aggregated hPSCs with a simplified blastocyst induction medium (“BIM”). Also provided are systems for extended culture of human blastoids on a 3D matrix, including blastoids generated from naïve hPSCs according to methods described herein.

### CROSS-REFERENCE TO RELATED APPLICATIONS

This application claims priority from U.S. Provisional Application Ser. No. 63/489,628 filed on 10 Mar. 2023, which is incorporated herein by reference in its entirety.

### STATEMENT REGARDING FEDERALLY SPONSORED RESEARCH OR DEVELOPMENT

Not applicable.

### FIELD

The present disclosure generally relates to extending human blastoid culture duration, quality, and yield on 3D surfaces.

### BACKGROUND

Blastoids (i.e., blastocyst-like structures) from human pluripotent stem cells (hPSCs) offer an exciting in vitro model system of human pre-implantation development, but important questions have been raised about the extent to which they faithfully mimic cell types in the human blastocyst. In particular, certain blastoid subpopulations originally identified as primitive endoderm or trophectoderm may actually correspond to amnion or extraembryonic mesoderm, which represent other extraembryonic lineages that emerge once the embryo has implanted. Thus far, blastoids have been generated from naïve hPSCs derived in two alternative culture conditions: PXGL and 5i/L/A. However, the original study describing blastoid generation from 5i/L/A naïve hPSCs reported a low efficiency of cavitation and a significant fraction of unaligned cell types. Additionally, while several attempts have been made to model implantation stages by transferring blastoids to tissue culture plastic or a thin coating of extracellular matrix (ECM), the long-term developmental potential of these structures remains unresolved.

### BRIEF DESCRIPTION OF THE DISCLOSURE

As described herein, this is the first disclosure of developed blastoid growth methods that supports extended human blastoid development for up to 21 days of in vitro culture. Previous groups have grown blastoids up to 13 days, including for 4 days of post-implantation culture (which corresponds roughly to day 10-11 human embryos) and 8 days total culture. All previous groups used a thin two-dimensional coating of extracellular matrix, as opposed to the 3D matrix described and utilized herein, and previous groups' blastoids lacked the structural complexity of the 3D-cultured blastoids of the present disclosure. In other words, the 3D matrix embodiments disclosed herein supported significantly enhanced blastoid development over previous work. As disclosed herein, substantial modifications were made to the blastoid induction medium to enhance the efficiency of blastoid development from previous protocols (5-10%) to up to 90% using the protocol disclosed herein. The finalized publication of the present disclosure entitled “3D-cultured blastoids model human embryogenesis from pre-implantation to early gastrulation stages” to Karvas et al. (2023), Cell stem cell, v. 30(9), pp. 1148-1165, and all associated supplemental material is herein incorporated by reference as support of the present disclosure.

Among the various aspects of the present disclosure is the provision of technology that significantly improves blastoid quality/yield in 5i/L/A media and compares very favorably with PXGL-derived blastoids. Several groups have reported attachment of blastoids to flat surfaces which collapsed within several days. The synthetic human blastoids of the present disclosure enabled culture for up 18 days in vitro, which far exceeds the current state of the art in literature. Further, the present disclosure describes synthetic human blastoid attachment to 3D matrices for the duration of in vitro culture.

In one aspect of the present disclosure a method for generating blastoids from naïve hPSCs is provided. The method comprises: exposing the naïve hPSCs to 5i/L/A; aggregating the exposed hPSCs in N2B27; and treating the aggregated hPSCs with a simplified blastocyst induction medium (“BIM”).

In some embodiments, the exposing is carried out for about 1 day; the aggregating is carried out for about 2 days; and the treating is carried out for about 5 days. In some embodiments, the naïve hPSCs are WIBR3 OCT4-GFP naïve hPSCs. In some embodiments, a GSK3α/β inhibitor (such as CHIR99021), a BRAF inhibitor (such as SB590885), a leukemia inhibitory factor (LIF), an Activin/Nodal/TGFβ inhibitor (such as SB431542), and/or a combination of Activin and FGF2 is/are omitted. In some embodiments, an amount of A83-01, an amount of sodium pyruvate, and/or a seeding density is increased.

In another aspect of the present disclosure a system for extended culture of human blastoids from naïve hPSCs is provided. The system comprises: blastoids generated from naïve hPSCs by a method comprising exposing the naïve hPSCs to 5i/L/A, aggregating the exposed hPSCs in N2B27, and treating the aggregated hPSCs with a simplified blastocyst induction medium (“BIM”); and a 3D matrix.

In some embodiments, the exposing is carried out for about 1 day; the aggregating is carried out for about 2 days; and the treating is carried out for about 5 days. In some embodiments, the naïve hPSCs are WIBR3 OCT4-GFP naïve hPSCs. In some embodiments, the 3D matrix comprises Cultrex, Matrigel, or a combination of Cultrex and Matrigel.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1(A-G) is an exemplary embodiment of efficient blastoid formation from naïve hPSCs derived in 5i/L/A in accordance with the present disclosure. FIG. 1A: Schematic representation of the blastoid formation protocol. Naïve hPSCs were seeded into Aggrewell-400 plates and allowed to aggregate in 5i/L/A naïve media for 1 day. The media were changed to N2B27+NaPyruvate (NaPy) for an additional 2 days to allow release from pluripotency. The media were then changed to BIM for an additional 4-5 days to promote the specification of TE, PE, and the formation of the blastocoel cavity. The predominant media components (with inhibitor targets) are indicated on the right. FIG. 1B: Time course images of 3 representative blastoids generated from WIBR3 OCT4-GFP naïve hPSCs over 7 days of culture. Cavitation occurs during the last 1-2 days. FIG. 1C: Maximum projection immunofluorescence (IF) confocal imaging of a WIBR3 OCT4-GFP blastoid at D7. OCT4 (green) indicates epiblast (EPI), GATA3 (orange) marks surrounding trophectoderm (TE), and SOX17 (magenta) marks primitive endoderm (PE). Scale bar=100 μm. FIG. 1D: Maximum projection IF images of WIBR3 blastoids indicate additional markers of EPI and TE. EPI markers include OCT4, SOX2, and IF116 (green). TE markers include CGA, TEAD1, NR2F2, ZO-1 (green), and nuclear YAP (red). Scale bar=250 μm.

FIG. 1E: Analysis of ICM position and blastocoel cavity formation performed with optical coherence tomography imaging (OCT) over 6 days of blastoid formation from WIBR3 OCT4-GFP naïve hPSCs. The top row of images shows a top-down-view of aggregates proliferating between D2-4 with cavitation occurring at D6. The red dotted line indicates the transverse plane used to visualize the side-view of the blastoids forming blastocoel cavities at six days. Scale bar=400 μm. FIG. 1F: OCT image of a single cavitated blastoid. The red dotted line highlights the ICM. The transverse section highlighted in green shows a densely-packed, localized ICM surrounded by TE and a blank inner blastocoel cavity. The yellow perpendicular plane additionally highlights the empty blastoid cavity interior. FIG. 1G: Quantification of ICM position within individual microwells. Data were quantified among 3 separate regions, total number of blastoids counted=141. [See also FIG. 2(A-H).]

FIG. 2(A-H) is an exemplary embodiment of optimization of naïve blastoid aggregation conditions [related to FIG. 1(A-G)] in accordance with the present disclosure. FIG. 2A: Characteristics data table for Conditions 1-8. FIG. 2B: PE and TE development in inverted-pyramidal Aggrewells for Conditions 1-8. FIG. 2C: Blastoid cavitation rates for Conditions 1-8. FIG. 2D: TE expansion based on GFP reporter activity. FIG. 2E: ICM area, blastoid area, and ICM:blastoid ratio. FIG. 2F: Blastoid cavitation rates for multiple naïve hPSC lines. FIG. 2G: Immunofluorescence (IF) analysis of generated blastoid expression. FIG. 2H: Blastoid diameter.

FIG. 3(A-J) is an exemplary embodiment of single cell transcriptome analysis of pre-implantation blastoids (D7) in accordance with the present disclosure. FIG. 3A: Single cell RNA-sequencing (scRNA-seq) of blastoids generated from WIBR3 OCT4-GFP naïve hPSCs at D7 reveals 4 major clusters by Uniform Manifold Approximation and Projection (UMAP) analysis: epiblast (EPI), primitive endoderm (PE), trophectoderm (TE), and polar trophectoderm (polar TE). FIG. 3B: Dotplots indicate gene expression patterns in the four distinct clusters shown in FIG. 3A. Average gene expression levels and the percentage of cells that express each gene are presented with differential color intensities and circle sizes, respectively. Red boxes highlight genes highly expressed within each cell type cluster. FIG. 3C: UMAP plots indicating expression of EPI (NANOG, POU5F1, SUSD2), PE (SOX17, PDGFRA, GATA4), TE (CDX2, GATA3, TEAD1), polar TE (CCR7, NR2F2, CGA), amnion (AME) (GABRP, ISL1, HEY1), and extraembryonic mesoderm (EXM) (VIM, POSTN, ANXA1). FIG. 3D: UMAP of scRNA-seq velocity trajectory analysis of D7 blastoids. Arrows indicate trajectory inferences of future cell states based on RNA Velocity. FIG. 3E: Diffusion map of scRNA-seq velocity trajectory analysis of D7 blastoids. Arrows indicate trajectory inferences of future cell states based on RNA Velocity. FIG. 3F: Latent time analysis based on RNA velocity indicates the temporal relationships between individual cells. FIG. 3G: Integration of D7 blastoid scRNA-seq dataset (Left) with scRNA-seq analysis of human IVF-derived embryos between days E3-E7 from Petropoulos et al. (Right). Note that D7 blastoids overlapped closely with cell types in E6-7 blastocysts, but not with earlier stages of human embryogenesis (E3-5). FIG. 3H: Merged UMAP of integrated D7 blastoid and E3-7 human embryo dataset. Note that blastoid EPI clustered with blastocyst EPI, blastoid PE clustered with blastocyst PE, blastoid TE clustered with blastocyst TE and mural TE, while blastoid polar TE clustered with blastocyst polar TE.

FIG. 3I and FIG. 3J: Integration of D7 blastoid (FIG. 3I) scRNA-seq dataset with scRNA-seq analysis of 3D-cultured human embryos (E6-14, FIG. 3J) from Xiang et al. Note that blastoid EPI, PE, and TE clusters overlapped closely with blastocyst EPI, PE (hyphoblast), and pre-CTB clusters, respectively. As expected, D7 blastoids did not contain a primitive streak (PS) cluster. [See also FIG. 4(A-D).]

FIG. 4(A-D) is an exemplary embodiment of extended characterization of pre-implantation blastoid single cell transcriptome data [related to FIG. 3(A-J)] in accordance with the present disclosure. FIG. 4A: cDNA libraries for single cell RNA-sequencing (scRNA-seq). FIG. 4B: Cellular composition of blastoids. FIG. 4C: Integration of scRNA-seq datasets from human embryos showing blastoid cluster alignment with counterparts human blastocysts. FIG. 4D: Transcriptional correspondence of polar TE cells in D7 blastoids with AME.

FIG. 5(A-M) is an exemplary embodiment of epiblast and trophoblast structures in 3D-cultured human blastoids (D14-18) in accordance with the present disclosure. FIG. 5A: Experimental strategy for examining the capacity of human blastoids to recapitulate early post-implantation development on 2D and 3D matrices. FIG. 5B: Brightfield images of blastoids that were derived from WIBR3 OCT4-GFP naïve hPSCs and maintained on either a thin coating of ECM (Top) or a thicker 3D ECM (Bottom) from D7-14. FIG. 5C: IF analysis of post-implantation lineages in human blastoids cultured on a thin coating of ECM (2D). DAPI in cyan marks all nuclei, OCT4 (green) marks EPI, GATA4 (red) marks PE, and KRT7 (magenta) marks trophoblast derivatives. The central plane (Right, featured box in white) displays an amniotic cavity (a.c.) forming within the EPI compartment. FIG. 5D: IF analysis of post-implantation lineages in human blastoids cultured on a thicker type of ECM (3D). DAPI in cyan marks all nuclei, OCT4 (green) marks EPI, GATA4 (red) marks PE, and KRT7 (magenta) marks trophoblast derivatives. The central plane (Right, featured box in white) displays an amniotic cavity within the EPI compartment (a.c.) and a cavity within the PE indicative of an emerging yolk sac cavity (y.c.). FIG. 5E: Protein expression and placental structures in 3D-cultured blastoids at D14. MMP2 (red) marks invasive trophoblasts, OCT4 (green) marks EPI, and GATA6 (magenta) marks PE. This image is a maximum projection of a 3D-cultured human blastoid (WIBR3 OCT4-GFP background). FIG. 5F: 3D-cultured human blastoids at D14 exhibit a large multinucleated syncytium marked by hCG-beta (red), while OCT4 (green) marks the EPI compartment. This image shows a single plane through the center of the primitive syncytium (PrSyn) structure. The white outline in the left image is featured in the right panel. FIG. 5G: (Left) Brightfield image of a D8 blastoid attached to the surface of the 3D ECM via its polar TE. Note that the mural TE is in the distance, out of the focal plane of the ICM and Polar TE. (Center) Brightfield image of a D10 blastoid following attachment and collapse of the TE upon 3D ECM. (Right) Brightfield image of a blastoid maintained on 3D ECM until D14. Note that D14 blastoids feature complex post-implantation trophoblast structures, including invasive primary villi (PV), and early forming secondary villous-like structures (SVL). Scale=200 μm. FIG. 5H: IF confocal maximum projection images of a D14 blastoid (WIBR3 OCT4-GFP background) cultured on 3D ECM marked with DAPI (blue, nuclei), OCT4 (green, EPI), and GATA4 (red, PE) (Left). Maximum projection of the left panel including KRT7 (white, trophoblast) (Middle). Center plane featuring the central portions of a secondary villous-like (SVL) structure lined with KRT7 and migratory primary villi (PV) (Right). Scale=200 μm. FIG. 5I: Extended 3D culture of WIBR1 blastoids until D18 resulted in expansion of invasive primary villi with co-localization of HLA-G (green) and CGB (red). Scale=200 μm. FIG. 5J: Secondary villous-like structures (SVL) protrude from the edge of the embryonic center and were lined with trophoblasts expressing KRT7 (red). Scale=200 μm. FIG. 5K: 3D-cultured WIBR3 OCT4-GFP blastoids showed a limited number of VIM+ extraembryonic mesoderm (EXM) cells arising near the OCT4-GFP+(EPI) region at D14. KRT7 (red) marks trophoblast cells at the periphery. FIG. 5L: Extended 3D culture of WIBR3 OCT4-GFP blastoids resulted in expansion of EXM cells marked by VIM (magenta, Left) and their migration into KRT7-positive trophoblast cells (red, Middle). Merged maximum projection image shows the integration of VIM+ cells into the trophoblast compartment by D18 (Right). FIG. 5M: An optimized 3D matrix promotes proportionate development of post-implantation lineages in D14 blastoids. Culture on 100% Geltrex resulted in a larger EPI (OCT4, green) compartment and a smaller trophoblast (KRT7, red) compartment (Left, maximum projection), while culture on 100% Matrigel resulted in the expansion of the trophoblast compartment and reduction of the EPI (Right, maximum projection). The graphs in the middle indicate the volume of each compartment (EPI, PE, and trophoblast) when Geltrex and Matrigel are mixed in various ratios (100%-0%, 75%-25%, 50%-50%) in D14 blastoids. [See also FIG. 6(A-C).]

FIG. 6(A-C) is an exemplary embodiment of evaluation of different media and matrices for post-implantation blastoid culture [related to FIG. 5(A-M)] in accordance with the present disclosure. FIG. 6A: IF analysis showing N2B27 vs IVC media. FIG. 6B: IF analysis showing 100% Geltrex vs 100% Matrigel. FIG. 6C: IF analysis showing Geltrex and Matrigel mixed in various ratios (100%-0%, 75%-25%, 50%-50%).

FIG. 7(A-G) is an exemplary embodiment of single cell transcriptome analysis of 3D-cultured human blastoids (D14) in accordance with the present disclosure. FIG. 7A: scRNA-seq analysis of 3D-cultured human blastoids (WIBR3 OCT4-GFP background) at D14. UMAP analysis revealed 6 clusters: EPI, PE, cytotrophoblast (CTB), syncytiotrophoblast (STB), primitive syncytium (PrSyn), and extravillous trophoblast (EVT). FIG. 7B: Dotplots indicate gene expression patterns in the clusters shown in FIG. 7A. Average gene expression levels and the percentage of cells that express each gene are presented with differential color intensities and circle sizes, respectively. Red boxes highlight genes highly expressed within each cell type cluster. FIG. 7C: UMAP plots indicating expression of EPI (SOX2, SFRP2), PE (GATA6, PDGFRA), CTB (ITGA6, YAP1), EVT (ASCL2, HLAG), PrSyn (ITGA1, CYP19A1), and STB (CGB5, SDC1). FIG. 7D: UMAP (Left) and diffusion analysis (Right) of scRNA-seq velocity trajectory analysis of D7 blastoids. Arrows indicate trajectory inferences of future cell states based on RNA Velocity. FIG. 7E: Integration of D14 blastoid scRNA-seq dataset with single cell analysis of 3D-cultured human embryos (E6-14) from Xiang et al. Note that all subpopulations identified in human embryos are represented in 3D-cultured blastoids at D14, except for primitive streak anlage (PsaEPI) and amnion (AME/AMEepi). FIG. 7F:UMAP projection of D14 blastoid data with a focus on the EPI and PE clusters featuring TBXT, MIXL1, PDGFRA, SOX17, SOX2, and VIM. FIG. 7G: Center plane through a confocal IF image of a blastoid at D18 showing DAPI (nuclei), SOX2 (remaining EPI), T/BRACHYURY (arrow indicates localization), and the merged image (Right). [See also FIG. 8(A-C).]

FIG. 8(A-C) is an exemplary embodiment of extended characterization of post-implantation blastoid single cell transcriptome data [related to FIG. 7(A-G)] in accordance with the present disclosure. FIG. 8A: UMAP analysis of D14 major clusters (PE, EPI, PrSyn, CTB, EVT, STB). FIG. 8B: UMAP analysis scatter plot showing nFeature RNA vs percent. FIG. 8C: UMAP plots indicating expression of Early mesoderm (TBXT, MIXL1), EXM (VIM), Amnion (ISL1, GABRP), and Primitive Syncytium (MMP2).

FIG. 9(A-F) is an exemplary embodiment of integrated single cell analysis reveals trajectories and driver genes during human blastoid development from D7 to D14 in accordance with the present disclosure. FIG. 9A: UMAP analysis showing the integration of D7 and D14 blastoid scRNA-seq data visualized in split plots (Left) or a merged plot (Right). FIG. 9B: Dotplot indicating gene expression patterns in the 10 distinct clusters from D7 and D14 shown in FIG. 9A. Average gene expression levels and the percentage of cells that express each gene are presented with differential color intensities and circle sizes, respectively. Red boxes highlight genes highly expressed within each cluster. FIG. 9C: Scatterplots for selected markers between D7 and D14 within integrated UMAP analysis. POU5F1 and GATA3 are consistently expressed in EPI and TE derivatives, respectively. On the other hand, SFRP2, THY1, and PODXL are dynamically expressed in the EPI clusters and TACSTD2, VGLL1, and SDC1 are dynamically expressed in trophoblast clusters. FIG. 9D: UMAP projection of scRNA-seq velocity trajectory analysis of integrated D7 and D14 blastoids. Arrows indicate trajectory inferences of future cell states based on RNA Velocity. FIG. 9E: Cell cycle analysis of integrated D7 and D14 datasets. The proportion of cells in G1, G2/M, and S phases are indicated based on cell cycle gene expression. FIG. 9F: Driver gene analysis by RNA Velocity. Highlighted driver genes include POU5F1 (EPI D7/D14), TCF7L1 (EPI D14), GATA6 and EPCAM (PE), ARID3A and VGLL1 (TE), SKP2 and TCF7L2 (CTB), GREB1L and KRT8 (EVT), and GCM1 and SDC1 (STB). Note that some of these driver genes display a steady RNA velocity state within the selected cell type (e.g., POU5F1 and KRT8), while other show a more dynamic state (e.g., GREB1L and TCF7L2). [See also FIG. 10(A-F).]

FIG. 10(A-F) is an exemplary embodiment of extended characterization of D7 and D14 integrated blastoid single cell transcriptome data [related to FIG. 9(A-F)] in accordance with the present disclosure. FIG. 10A: Integrated map analysis showing separated D7 and D14. FIG. 10B: Integrated map analysis of merged D7 and D14. FIG. 10C: 3D diffusion map of D7. FIG. 10D: 3D diffusion map of D14. FIG. 10E: 3D diffusion map of merged D7 and D14. FIG. 10F: Driver gene velocity and expression maps.

FIG. 11(A-E) is an exemplary embodiment of WNT signaling regulates lineage segregation during extended blastoid culture in accordance with the present disclosure. FIG. 11A: Scatterplots of genes related to canonical WNT signaling in D7 and D14 integrated scRNA-seq dataset, including CTNNB1, TCF7L1, TCF7L2, WNT3, WNT3A, and WNT7A. FIG. 11B: Human blastoids (WIBR3 OCT4-GFP background) were cultured on 3D ECM and treated with N2B27+E2 (control, left column), N2B27+E2+2 μM XAV939 (middle column), and N2B27+E2+2 μM CHIR99021 (right column) from D8 to D14. Brightfield (top row), OCT4-GFP signal (middle row), and merged (bottom row) images demonstrate blastoid morphology and OCT4-GFP localization. Scale=200 μm. FIG. 11C: Quantification of D14 blastoid features resulting from 2 μM XAV939 and 2 μM CHIR99021 treatment, including the ratio of OCT4-GFP area: total blastoid area normalized to the total average area (top left), OCT4-GFP signal intensity (top right), trophoblast projection area (bottom left), and overall blastoid area (bottom right). FIG. 11D: IF confocal imaging (max projections) of D14 blastoids treated with N2B27+E2 (control, left), N2B27+E2+2 μM XAV939 (middle), and N2B27+E2+2 μM CHIR99021 (right), showing DAPI (blue nuclei, top), OCT4 (green EPI, bottom), and KRT7 (magenta trophoblast, bottom). Scale=200 μm. FIG. 11E: Schematic summarizing the impact of WNT pathway modulation on extended human blastoid culture. Treatment with the tankyrase inhibitor XAV939 caused a reduction of the CTB shell and expansion of the EPI. On the other hand, treatment with the WNT activator CHIR99021 caused an expansion of the CTB shell and reduction of the EPI. Reduced trophoblast invasive projections were seen upon treatment with either XAV939 or CHIR99021. Blastoid features were analyzed at D14 of 3D culture after 6 days of inhibitor treatment.

FIG. 12 is an exemplary embodiment of a 3D gel matrix comparison of 100% Cultrex versus Cultrex+20% matrigel in accordance with the present disclosure.

### DETAILED DESCRIPTION OF THE DISCLOSURE

The present disclosure is based, at least in part, on the discovery that human blastoid culture duration, quality, and yield is improved based on novel, conditional protocols on 3D matrices.

The present disclosure provides efficient protocols for generating blastocyst-like structures (“blastoids”) from human pluripotent stem cells (hPSCs) and enabling their culture through early stages of human post-implantation development on a 3D extracellular matrix. Given ethical and practical constraints on accessing human embryos for research purposes, it has been nearly impossible to study the etiology of implantation failure and early pregnancy loss. Studies in recent years have shown that naïve hPSCs have the remarkable ability to self-organize into blastoids that model lineage segregation in the pre-implantation embryo. However, the extent to which blastoids can recapitulate defining features of human post-implantation development in vitro has remained unresolved.

As disclosed herein, blastoids cultured on thick 3D extracellular matrices capture several hallmarks of early post-implantation development, including epiblast lumenogenesis, rapid expansion and diversification of trophoblast lineages, and robust invasion of extravillous trophoblast cells. Single cell transcriptome profiling revealed key trajectories and driver genes up to day 14, while extended blastoid culture resulted in localized activation of the primitive streak marker BRACHYURY by day 18. It is also disclosed herein that modulation of WNT signaling alters cell fate decisions in post-implantation blastoids. The present disclosure demonstrates that 3D-cultured human blastoids model embryonic and extraembryonic lineage segregation during early post-implantation development and offers a window into a previously inaccessible stage of human embryogenesis. Opportunities emerging from this technology include the investigation of implantation failure and early pregnancy loss, screening for contraceptives, and the generation of more advanced human embryo models capable of undergoing gastrulation and germ cell induction.

According to the present disclosure, optimized conditions for blastoid generation from 5i/L/A naïve hPSCs and developed methodologies that support extended culture on 3D matrices are provided. Using single cell RNA sequencing, it is demonstrated herein that blastoids contain most lineages of the early post-implantation embryo by day 14, while extended culture results in the activation of primitive streak markers and more complex placental structures by day 18. This is the first demonstration that stem-cell-based embryo models can be cultured to such an advanced stage of human development and provides a novel platform to study a previously inaccessible stage of human embryogenesis that is associated with high rates of early pregnancy loss. The present disclosure also offers a stepping-stone for generating even more advanced embryo models capable of forming primordial germ cells and red blood cells, which will enable the generation of several therapeutically relevant cell types from hPSCs.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product-consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, signals can be modulated (e.g., reduced, eliminated, or enhanced) using genome editing.

As described herein, activity, signals, expression, or function can be modulated (e.g., reduced, eliminated, or enhanced) using genome editing (e.g., upregulate, downregulate, overexpress, underexpress, express (e.g., transgenic expression), knock in, knock out, knockdown).

Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage by genome editing can result in protection from various conditions and diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications to target cells by the removal or addition of signals (e.g., activation (e.g., CRISPRa), upregulation, overexpression, downregulation).

For example, the methods as described herein can comprise a method for altering a target polynucleotide sequence in a cell comprising contacting the polynucleotide sequence with a clustered regularly interspaced short palindromic repeats-associated (Cas) protein.

### Gene Therapy and Genome Editing

Gene therapies can include inserting a functional gene with a viral vector, and are rapidly advancing.

There has recently been an improved landscape for gene therapies. For example, in the first quarter of 2019, there were 372 ongoing gene therapy clinical trials (Alliance for Regenerative Medicine, 5/9/19).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

### Gene Therapy Strategies.

| | Strategy |
|---|---|
| Viral Vectors | |
| Retroviruses | Retroviruses are RNA viruses transcribing their |
| | single-stranded genome into a double-stranded DNA |
| | copy, which can integrate into host chromosome |
| Adenoviruses | Ad can transfect a variety of quiescent and |
| (Ad) | proliferating cell types from various species and |
| | can mediate robust gene expression |
| Adeno-associated | Recombinant AAV vectors contain no viral DNA and |
| Viruses (AAV) | can carry ~4.7 kb of foreign transgenic material. |
| | They are replication defective and can replicate |
| | only while coinfecting with a helper virus |
| Non-viral vectors | |
| plasmid DNA | pDNA has many desired characteristics as a gene |
| (pDNA) | therapy vector; there are no limits on the size or |
| | genetic constitution of DNA, it is relatively |
| | inexpensive to supply, and unlike viruses, |
| | antibodies are not generated against DNA in |
| | normal individuals |
| RNAi | RNAi is a powerful tool for gene specific silencing |
| | that could be useful as an enzyme reduction therapy |
| | or means to promote read-through of a premature stop |
| | codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing implantation failure and early pregnancy loss in a subject in need thereof by administration of a therapeutically effective amount of a chemical and/or cell composition, so as to promote embryonic implantation and retain early pregnancy.

Methods described herein are generally performed on a subject in need thereof. A subject in need of the therapeutic methods described herein can be a subject having, diagnosed with, suspected of having, or at risk for reversing implantation failure and/or early pregnancy loss. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a chemical and/or cell composition is, for example, an amount that would cause the desired therapeutic effect in a subject while minimizing undesired side effects. In various embodiments, an effective amount of a chemical and/or cell composition described herein can substantially inhibit, slow the progress of, or limit the development of conditions leading to implantation failure and early pregnancy loss.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a chemical and/or cell composition can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to prevent/avoid implantation failure and early pregnancy loss.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a chemical and/or cell composition can occur as a single event or over a time course of treatment. For example, a chemical and/or cell composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for implantation failure and early pregnancy loss.

A chemical and/or cell composition can be administered simultaneously or sequentially with another agent, such as an antibiotic, an anti-inflammatory, or another agent. For example, a chemical and/or cell composition can be administered simultaneously with another agent, such as an antibiotic or an anti-inflammatory. Simultaneous administration can occur through administration of separate compositions, each containing one or more of a chemical and/or cell composition, an antibiotic, an anti-inflammatory, or another agent. Simultaneous administration can occur through administration of one composition containing two or more of a chemical and/or cell composition, an antibiotic, an anti-inflammatory, or another agent. A chemical and/or cell composition can be administered sequentially with an antibiotic, an anti-inflammatory, or another agent. For example, a chemical and/or cell composition can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the chemical and/or cell composition may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a chemical and/or cell composition may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

### Also Provided are Screening Methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules database; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: 3D-Cultured Human Blastoids Model Lineage Segregation During Early Post-Implantation Development

The finalized publication of the present disclosure entitled “3D-cultured blastoids model human embryogenesis from pre-implantation to early gastrulation stages” to Karvas et al. (2023), Cell stem cell, v. 30(9), pp. 1148-1165, and all associated supplemental material is herein incorporated by reference as support of the present disclosure.

Naïve human pluripotent stem cells have the remarkable ability to self-organize into blastocyst-like structures (“blastoids”) that model lineage segregation in the pre-implantation embryo. However, the extent to which blastoids can recapitulate defining features of human post-implantation development in vitro remains unresolved. As disclosed herein, blastoids cultured on thick 3D extracellular matrices capture several hallmarks of early post-implantation development, including epiblast lumenogenesis, rapid expansion and diversification of trophoblast lineages, and robust invasion of extravillous trophoblast cells. Single cell transcriptome profiling reveals key trajectories and driver genes up to day 14, while extended blastoid culture results in the localized activation of the primitive streak marker BRACHYURY. Also demonstrated herein is the finding that modulation of WNT signaling alters cell fate decisions in post-implantation blastoids. This disclosure demonstrates that 3D-cultured human blastoids model embryonic and extraembryonic lineage segregation during early post-implantation development and offers a window into a previously inaccessible stage of human embryogenesis.

An aspect of the present disclosure describes a protocol for efficient blastoid formation from naïve hPSCs derived in 5i/L/A. Provisions of the present disclosure include: (i) 3D-cultured blastoids display trophoblast expansion, diversification, and invasion; (ii) single cell analysis reveals driver genes and trajectories in blastoids up to day 14; and (iii) WNT signaling regulates cell fate decisions during extended blastoid culture.

### Introduction

Human embryonic development requires the timely segregation between pluripotent cells that give rise to the fetus, as well as extraembryonic cells that mediate implantation into the uterus. However, information about this essential phase of human development remains limited as it takes place in utero and is therefore largely inaccessible to scientific investigation. In a transformative advance, several groups have attempted to bridge this gap and provide insights about these early developmental stages by developing embryo models from mouse and human stem cells in vitro. Initial studies in the mouse system aggregated embryonic and extraembryonic stem cells to create structures that resemble either the pre-implantation blastocyst (“blastoids”) or the post-implantation embryo. The fact that embryonic and extraembryonic stem cells must be combined in order to create mouse embryo models is consistent with lineage restriction by the mid-blastocyst (late 32-cell) stage of mouse development, such that pluripotent cells within the inner cell mass (ICM) can no longer contribute to the outer trophectoderm (TE) layer. Accordingly, mouse embryonic stem cells, which correspond to the pluripotent epiblast (EPI), require genetic perturbation to acquire extraembryonic fates. In contrast, it has been shown that naïve human pluripotent stem cells (hPSCs) have an extensive extraembryonic potential, readily giving rise to both TE and primitive endoderm (PE) derivatives in vitro. More recently, this unrestricted developmental potential has been leveraged by several groups to generate blastoids comprising all three lineages of the blastocyst solely from naïve hPSCs.

Blastoids offer an exciting in vitro model system of human pre-implantation development, but important questions have been raised about the extent to which they faithfully mimic their counterparts in the human blastocyst. In particular, certain blastoid subpopulations originally identified as PE or TE may actually correspond to amnion (AME) or extraembryonic mesoderm (EXM), which represent other extraembryonic lineages that emerge once the embryo has implanted. Thus far, blastoids have been generated from naïve hPSCs derived in two alternative culture conditions: PXGL and 5i/L/A. However, the original study describing blastoid generation from 5i/L/A naïve hPSCs reported a low efficiency of cavitation and a significant fraction of unaligned cell types. Additionally, while several groups attempted to model implantation stages by transferring blastoids to tissue culture plastic or a thin coating of extracellular matrix (ECM), the long-term developmental potential of these structures remains unresolved. Herein, the conditions have been optimized for blastoid generation from 5i/L/A naïve hPSCs and investigated their capacity for extended culture on a variety of thick 3D matrices, a strategy previously used to successfully culture human embryos through implantation stages. A detailed single cell transcriptome analysis was then performed to resolve cellular trajectories and define driver genes during blastoid development up to day 14 (D14). Our results reveal that 3D-cultured human blastoids model lineage segregation during early post-implantation development and provide a foundation for creating more advanced embryo models.

### Results

A Protocol for Efficient Blastoid Formation from Naïve hPSCs Derived in 5i/L/A

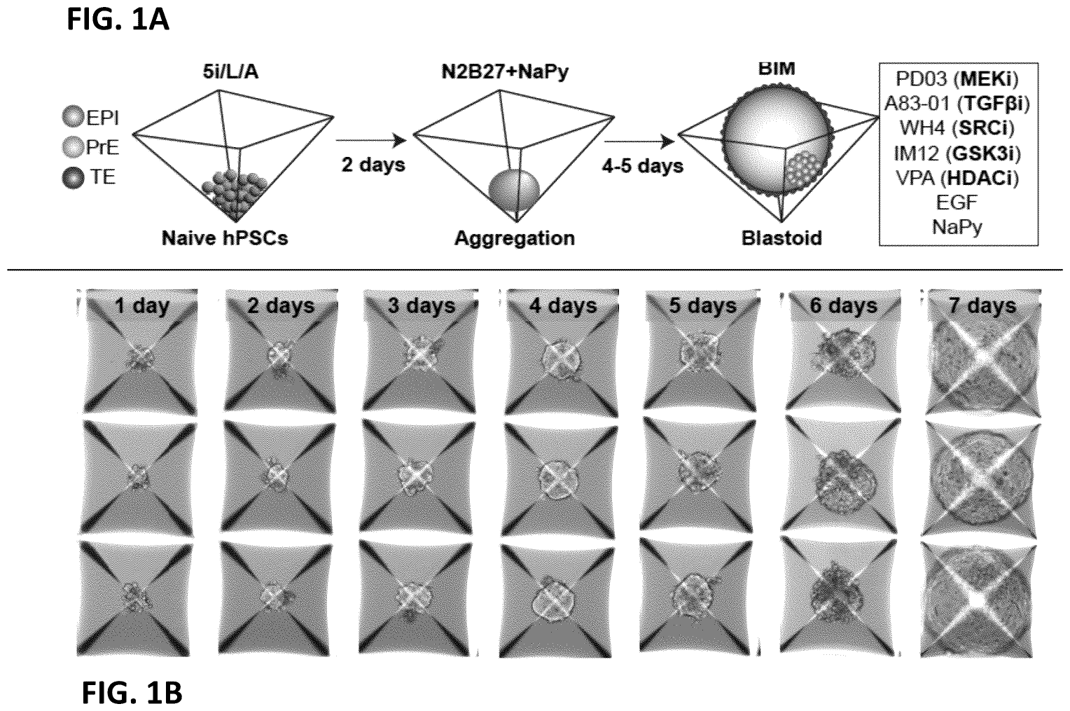

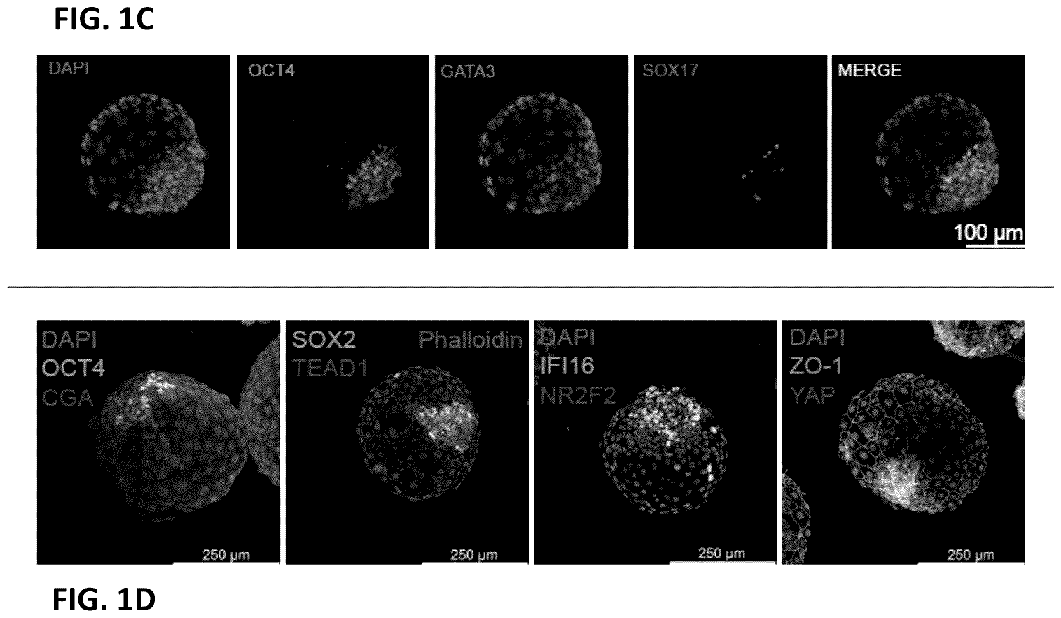

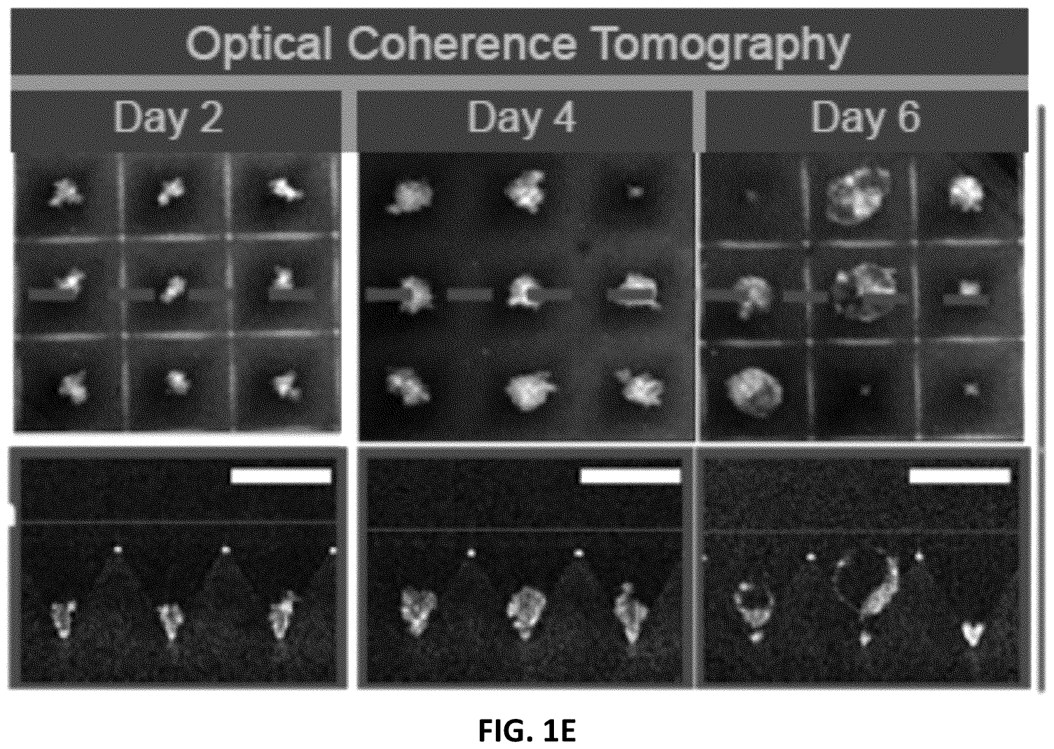

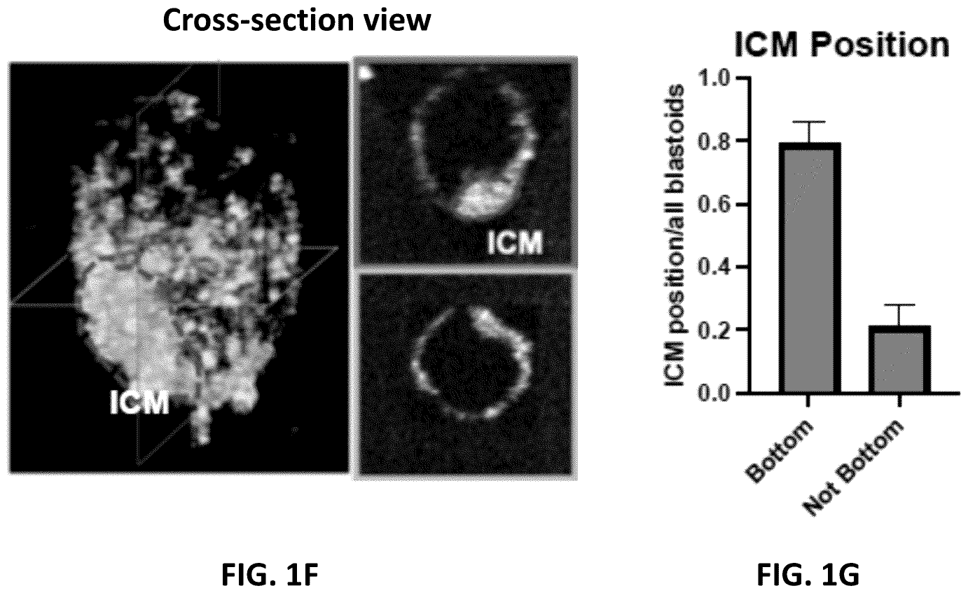

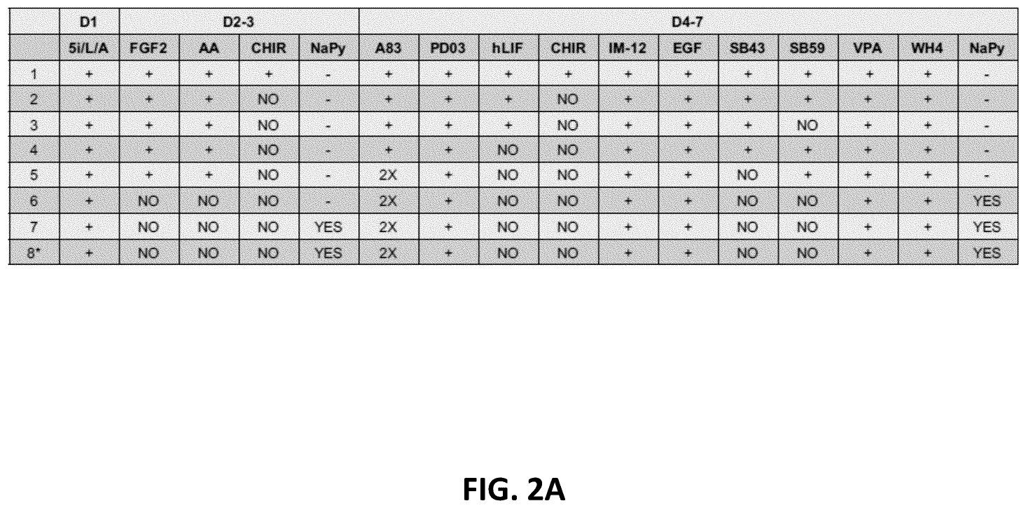

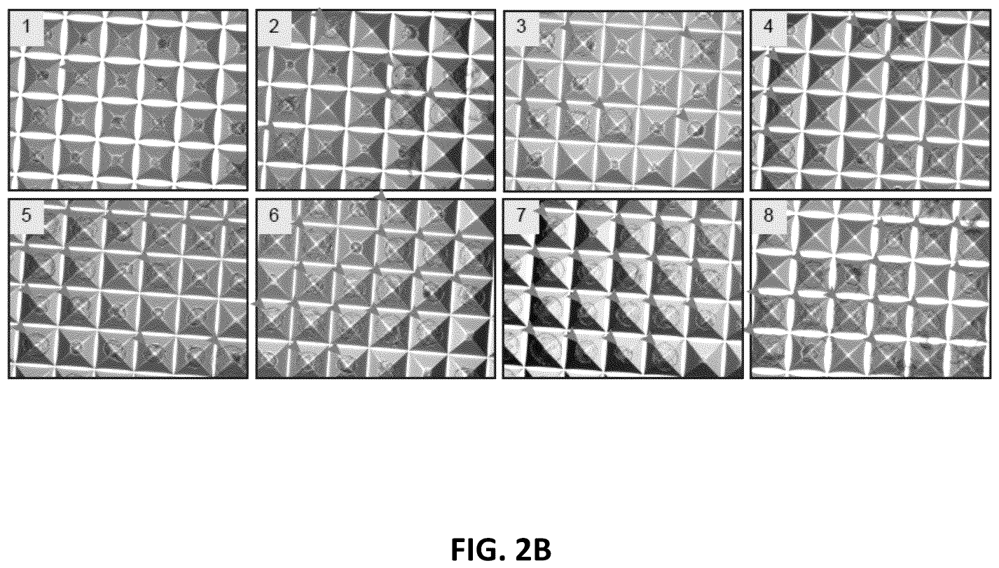

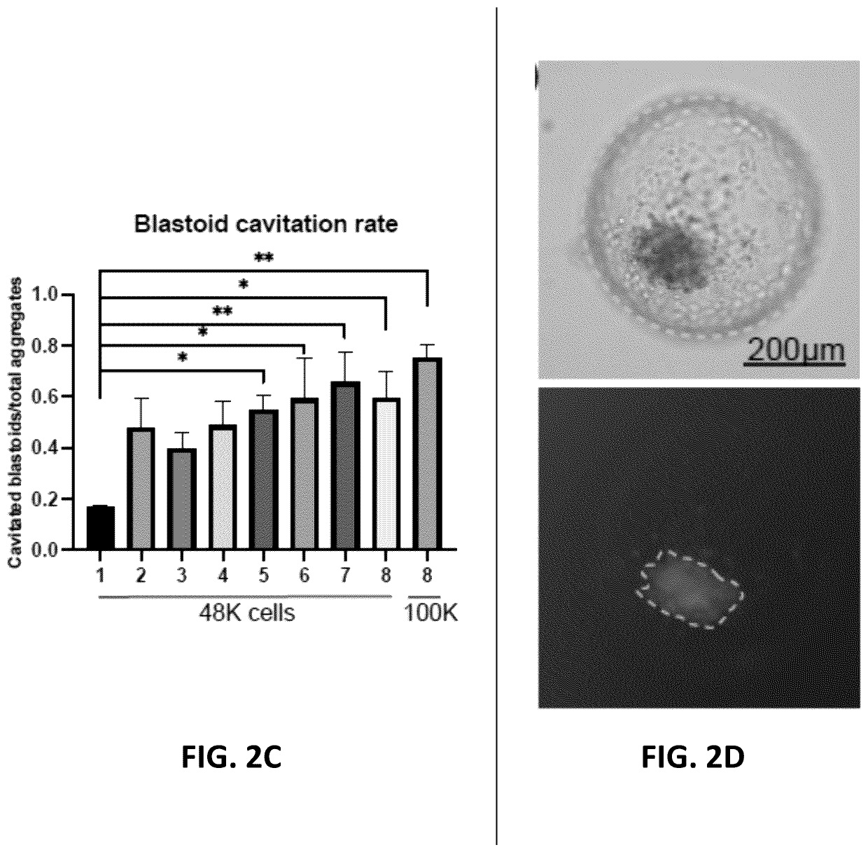

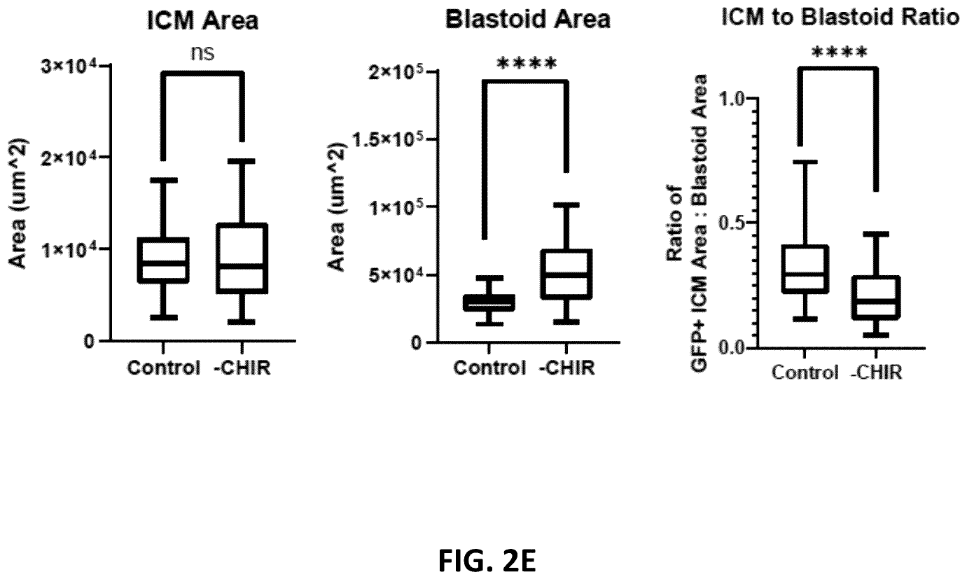

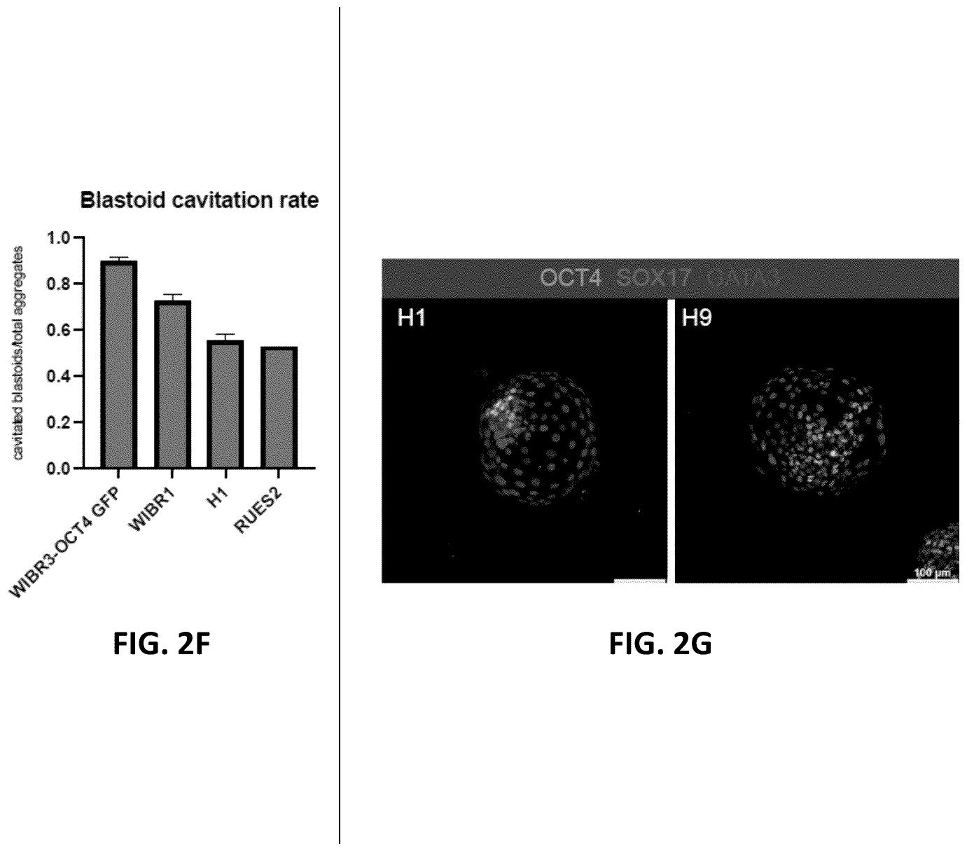

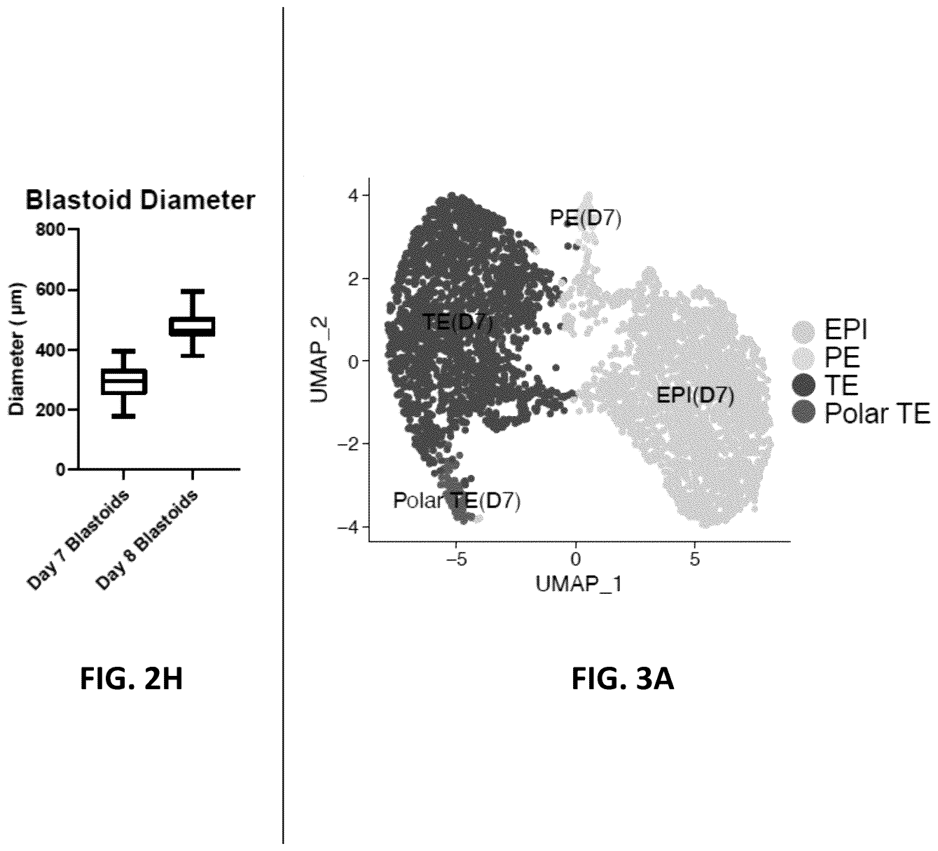

Blastoids were originally generated from 5i/L/A naïve hPSCs by sequential treatment with two media designed to promote PE and TE development in inverted-pyramidal Aggrewells. Using these conditions, cavitated structures were obtained from WIBR3 OCT4-GFP naïve hPSCs at low efficiency (˜5-10%), consistent with the original report (FIG. 2A-C, “Condition 1”). It was found that omission of the GSK3a/P inhibitor CHIR99021 enhanced the rate of cavitation (FIG. 2A-C “Condition 2”). By calculating the ICM:TE ratio based on OCT4-GFP reporter activity, it was concluded that omission of CHIR99021 specifically promotes TE expansion (FIG. 2D-E). Furthermore, the BRAF inhibitor SB590885 and leukemia inhibitory factor (LIF) could be removed without adversely affecting the rate of cavitation (FIG. 2A-C, “Conditions 3 and 4”). Also, the Activin/Nodal/TGFβ inhibitor SB431542 could be omitted by increasing the dose of A83-01, which targets the same pathway (FIG. 2A-C, “Condition 5”). Other modifications included the omission of Activin and FGF2 from days 2-3 (Condition 6), the addition of sodium pyruvate (NaPy, Condition 7), and a higher seeding density (Condition 8) (FIG. 2A-C). In summary, an efficient method has been established for blastoid formation from 5i/L/A naïve hPSCs that involves 1 day of exposure to 5i/L/A, 2 days of aggregation in N2B27, and 5 days of treatment with a simplified blastocyst induction medium (“BIM”) (FIG. 1A-C). These conditions achieved blastoid cavitation from multiple naïve hPSC lines with an efficiency up to 85% (FIG. 2F).

Immunofluorescence (IF) analysis confirmed that blastoids generated with this protocol express OCT4 in the EPI, GATA3 in the TE, and the PE marker SOX17 along the surface of the ICM that lines the blastocoel-like cavity (FIG. 1C and FIG. 2G). The overall diameter of D7 blastoids was 290±67 μm (FIG. 2H). While this is slightly larger than human IVF-derived blastocysts on average, a larger blastocyst diameter (>220 μm) correlates with higher live birth rates. When left in culture until D8, these blastoids continued to expand and eventually floated out of the Aggrewells (FIG. 2H). The presence of a well-developed TE epithelium was confirmed by IF staining with antibodies against CGA, TEAD1, NR2F2, and YAP (FIG. 1D). We also confirmed that ICM cells within blastoids express IF116, which was recently proposed as a marker of the human ICM (FIG. 1D). We then monitored the process of blastoid formation from naïve hPSCs in real time using optical coherence tomography (OCT), which allows for cross-sectional imaging (FIG. 1E). This analysis revealed that the ICM specifically develops on the side of the blastoid that is in contact with the bottom of the Aggrewell, suggesting a previously unappreciated biomechanical influence on ICM development (FIG. 1E-G).

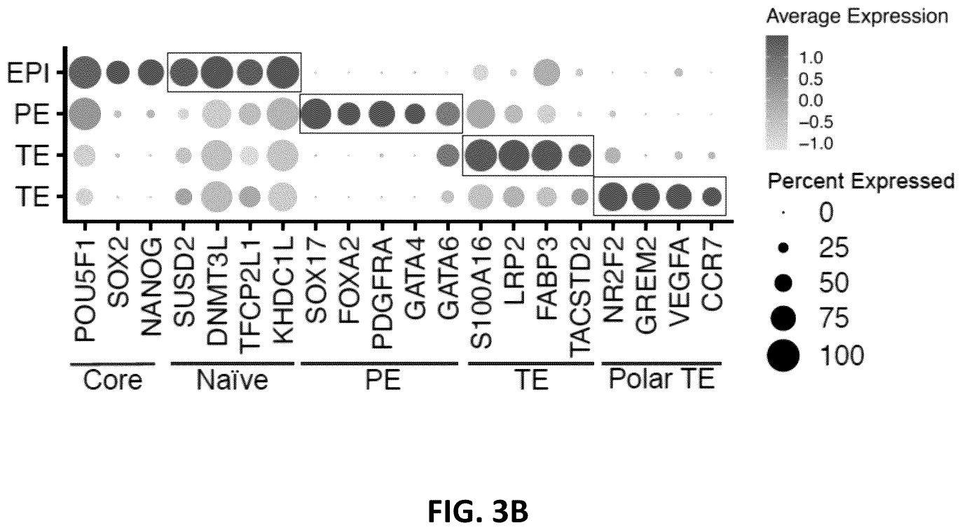

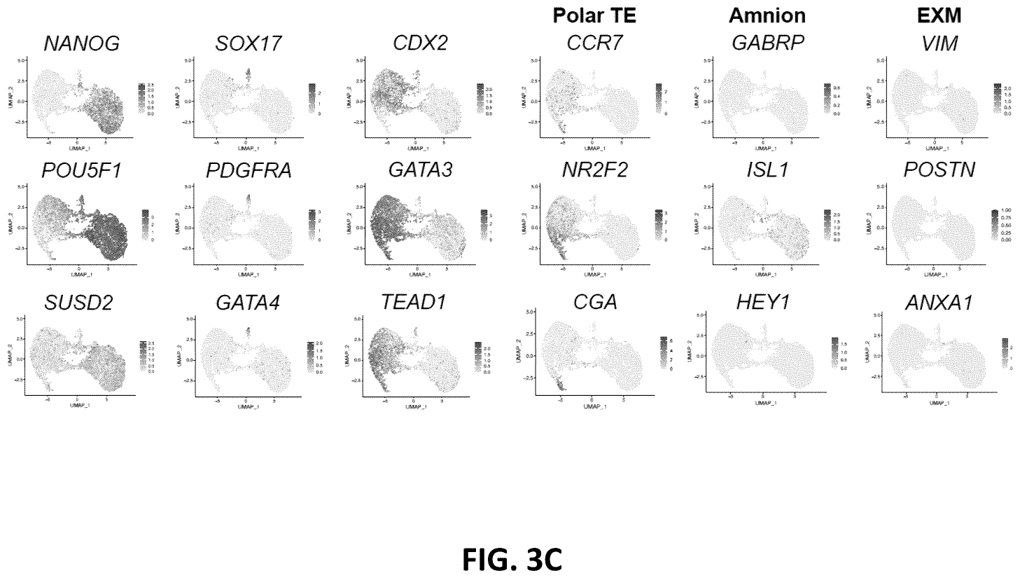

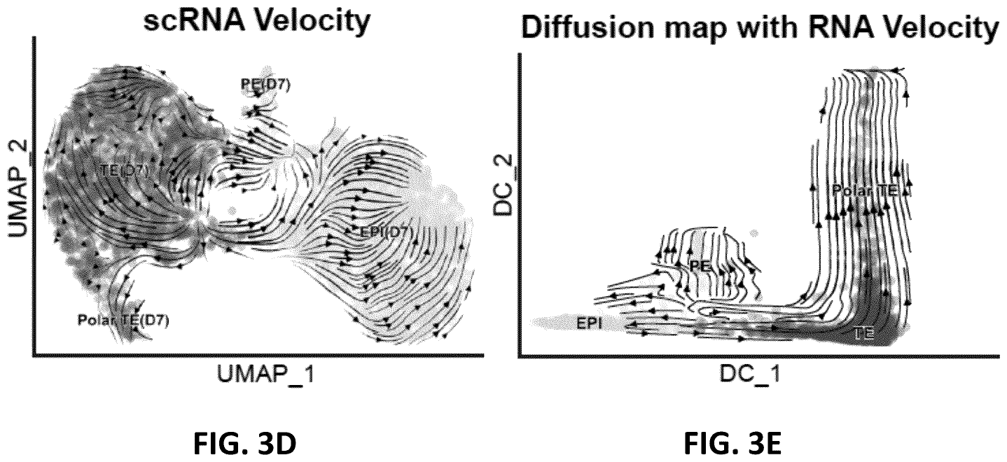

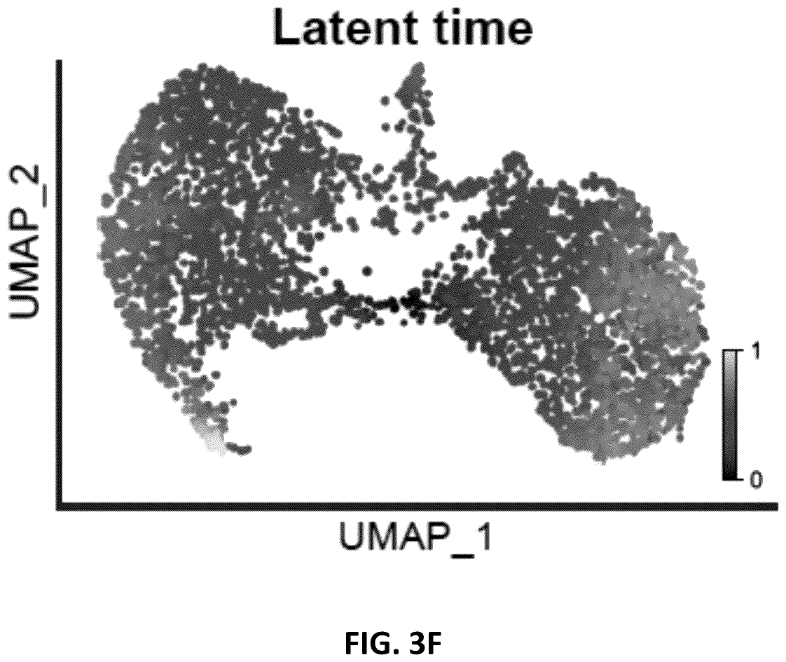

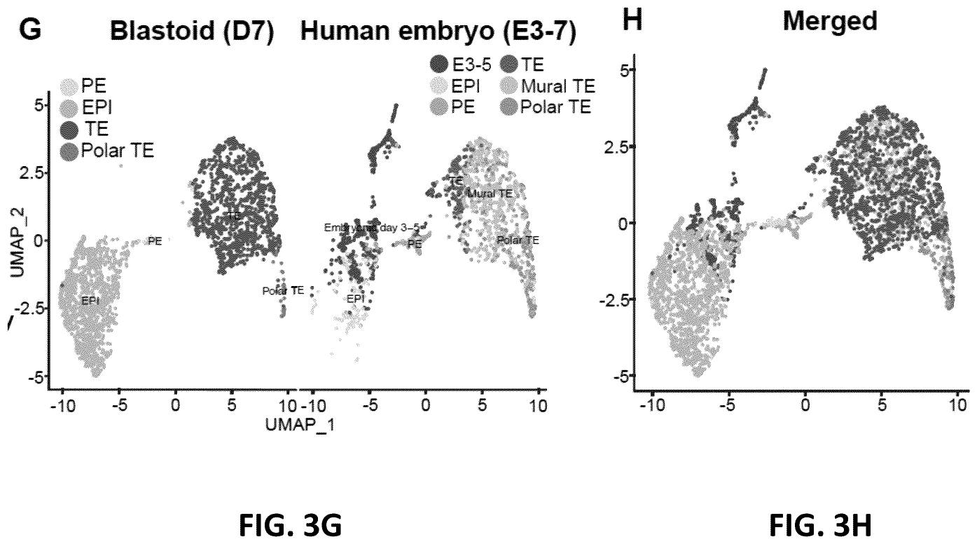

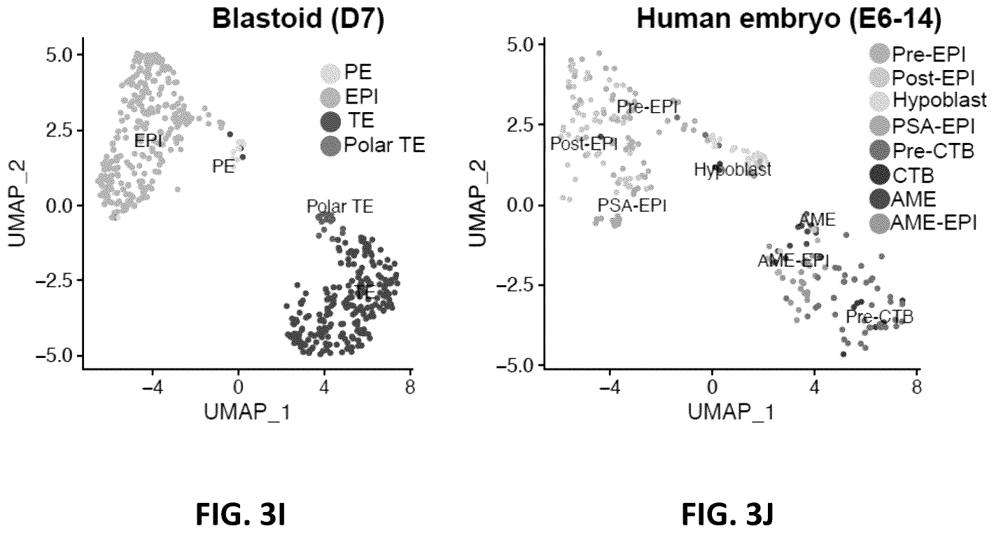

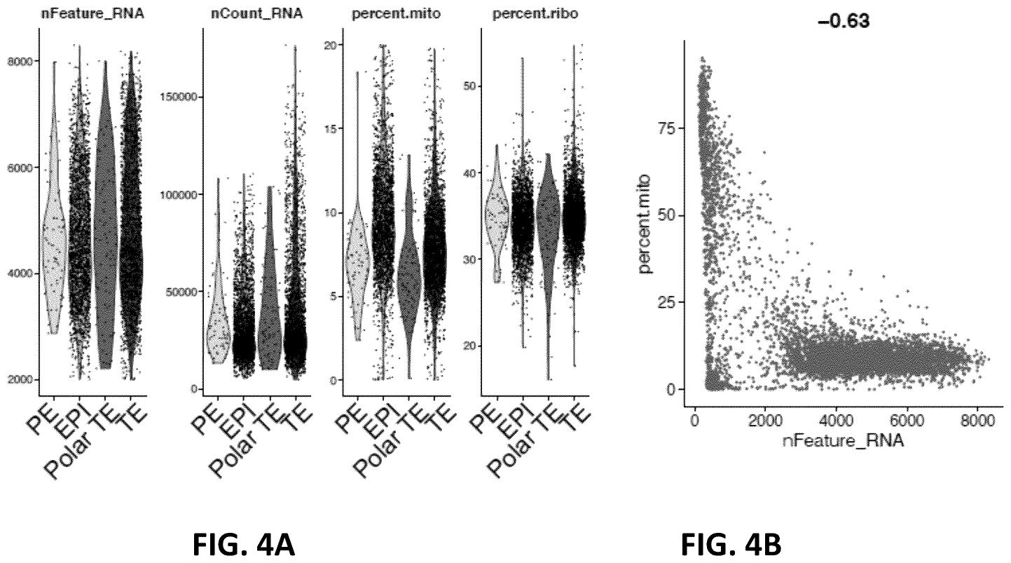

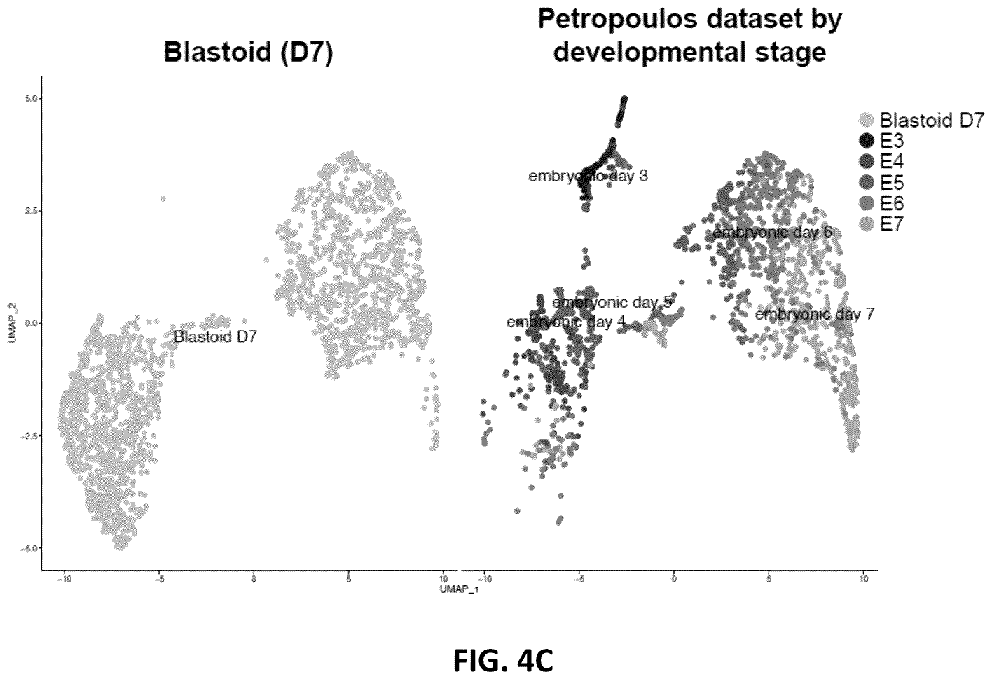

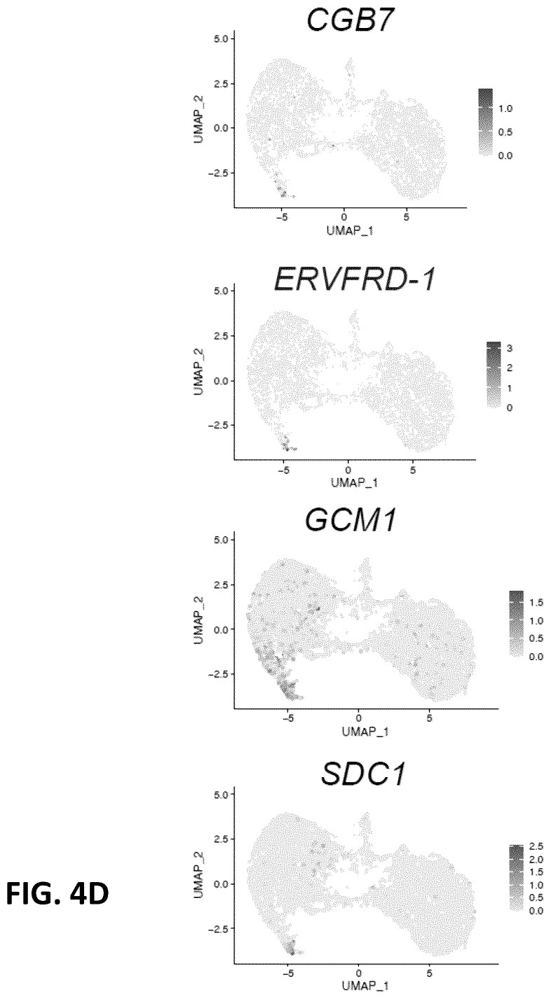

To examine the cellular composition of blastoids at D7, we dissociated them into single cells and generated cDNA libraries for single cell RNA-sequencing (scRNA-seq) using the 10× Genomics platform (FIG. 4A-B). Dimensional reduction analysis by Uniform Manifold Approximation and Projection (UMAP) revealed that D7 blastoids predominantly consist of two major clusters (EPI and TE) and two smaller clusters (PE and polar TE) (FIG. 3A). The EPI cluster was marked by elevated expression of genes such as OCT4, NANOG, DNMT3L, and SUSD2; the TE cluster was marked by high expression of CDX2, GATA2, GATA3, and TEAD1 (FIGS. 3B-C); the PE cluster was marked by SOX17, FOXA2, GATA4 and PDGFRA; and the polar TE cluster was marked by CCR7, NR2F2, and the mature TE marker CGA (FIGS. 3B-C). Diffusion map analysis revealed two major developmental trajectories: one that emerged from EPI into PE and another from TE into polar TE (FIGS. 3D-E). This analysis suggests that the PE emerges from the EPI, in agreement with recent observations in human embryos. Accordingly, analysis of latent time revealed a common progenitor of all blastoid populations within the EPI cluster, while the polar TE appeared to be most recent cluster (FIG. 3F). Importantly, D7 blastoids displayed negligible expression of AME or EXM markers, which have been identified as off-target cell types in other blastoid studies (FIG. 3C). Integration with two independent scRNA-seq datasets from human embryos revealed close alignment of EPI, TE, and PE clusters in blastoids with their counterparts in human blastocysts at embryonic day (E)6-7 (FIG. 3G-J and FIG. 4C). The cluster of polar TE cells in D7 blastoids shared some transcriptional correspondence with AME, which may be explained by their common syncytiotrophoblast (STB) signature (FIG. 40).

### Human Blastoids Cultured on Thick 3D Matrices Display EPI Lumenogenesis and Rapid Expansion and Diversification of Trophoblast Lineages

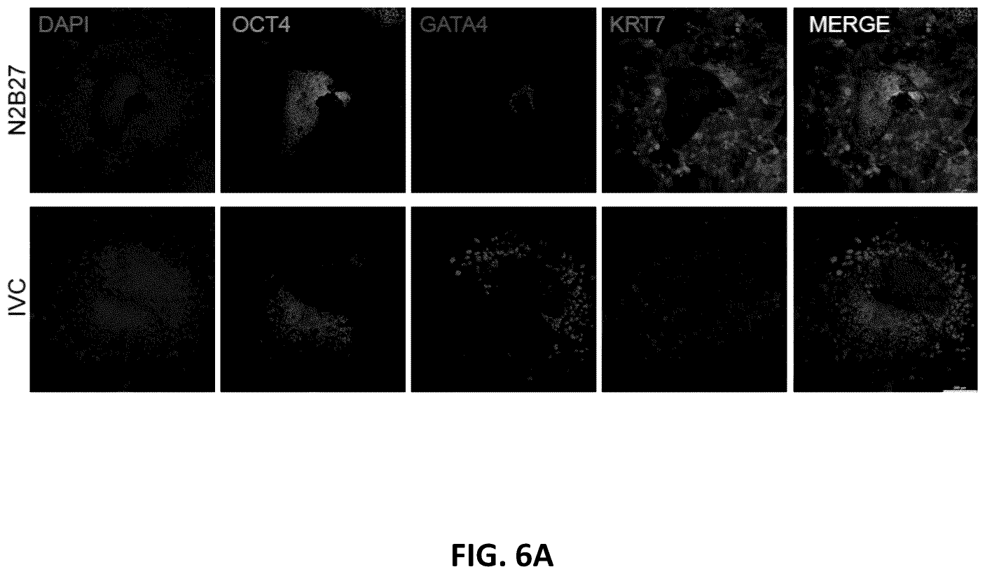

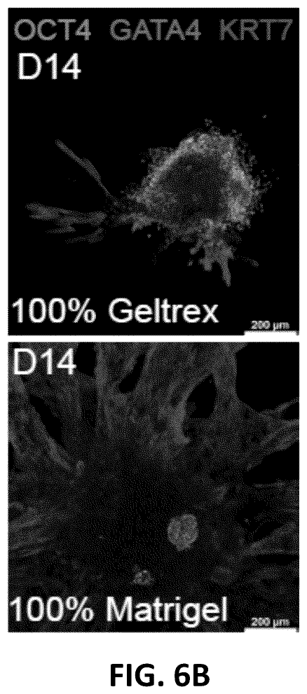

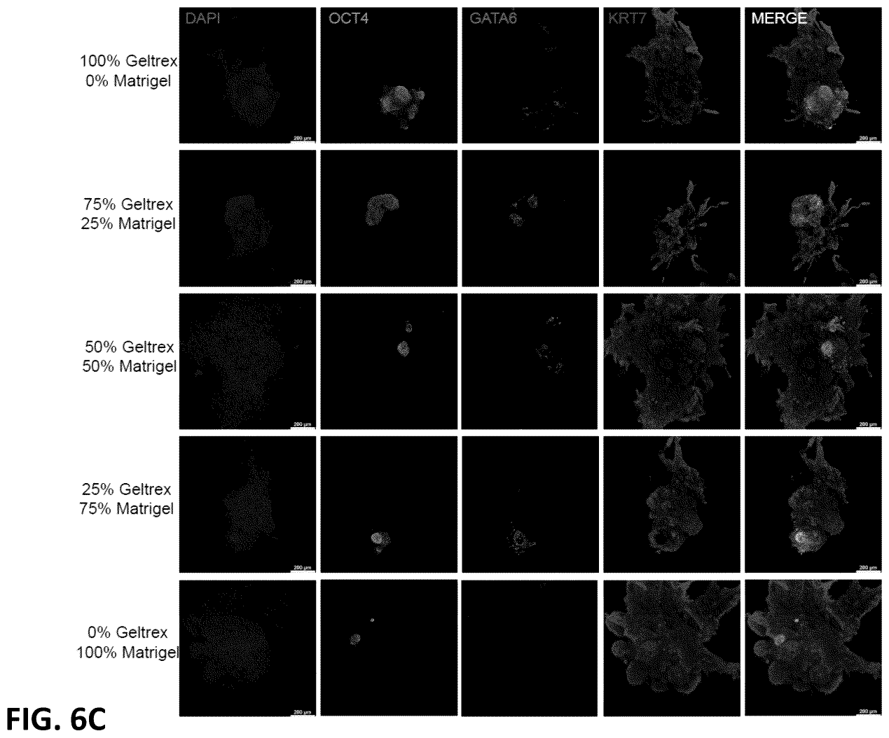

To examine the potential of naïve stem cell blastoids to model human post-implantation development, we transferred blastoids at D7 from Aggrewells to μ-slides coated with a thin layer of Geltrex, a commercially available ECM. We evaluated two alternative media for post-implantation blastoid culture: (i) advanced in vitro culture (IVC) media that support extended culture of human embryos; and (ii) serum-free N2B27 media, which were previously used for blastoid outgrowths. Blastoids were maintained in attachment cultures for 7 additional days (i.e., until D14 of total culture). IF analysis with antibodies against OCT4, GATA4, and KRT7 revealed that lineage-specific expression was maintained in N2B27, whereas post-implantation blastoids maintained in IVC media lacked KRT7-positive trophoblast cells (FIG. 6A).

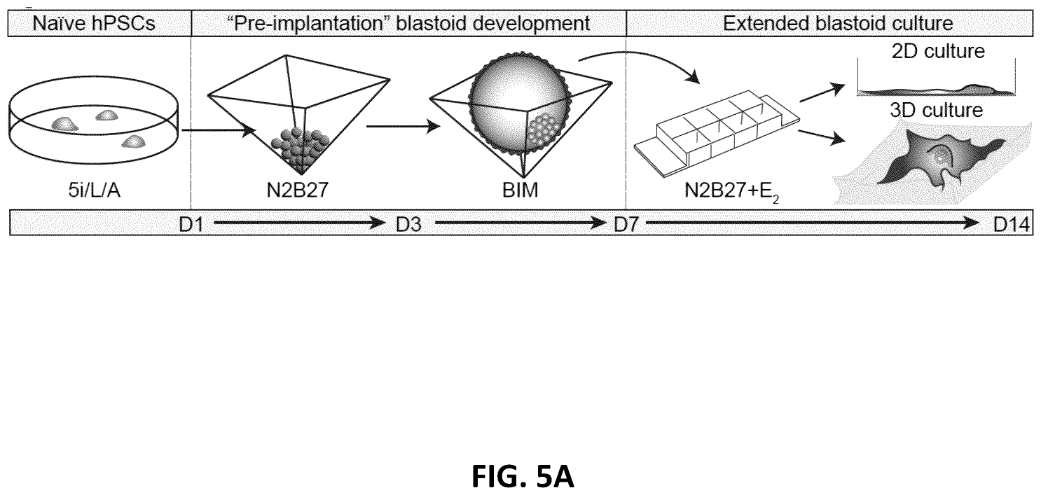

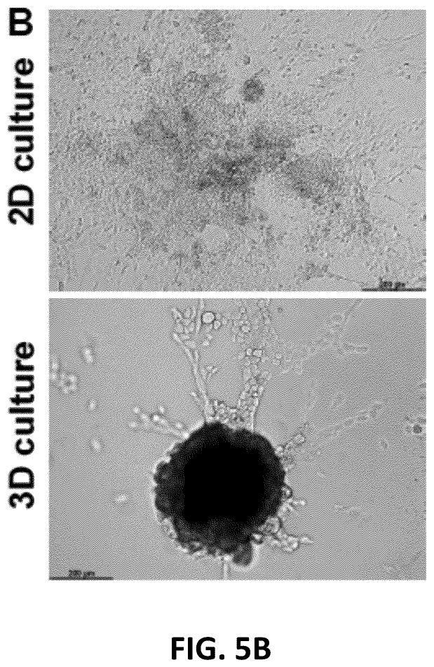

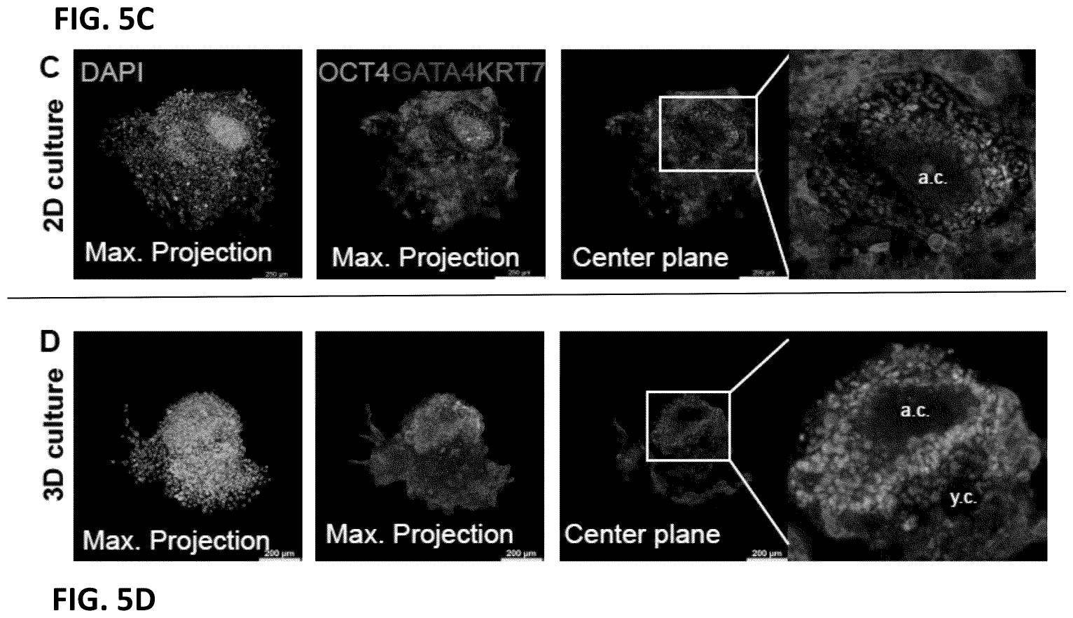

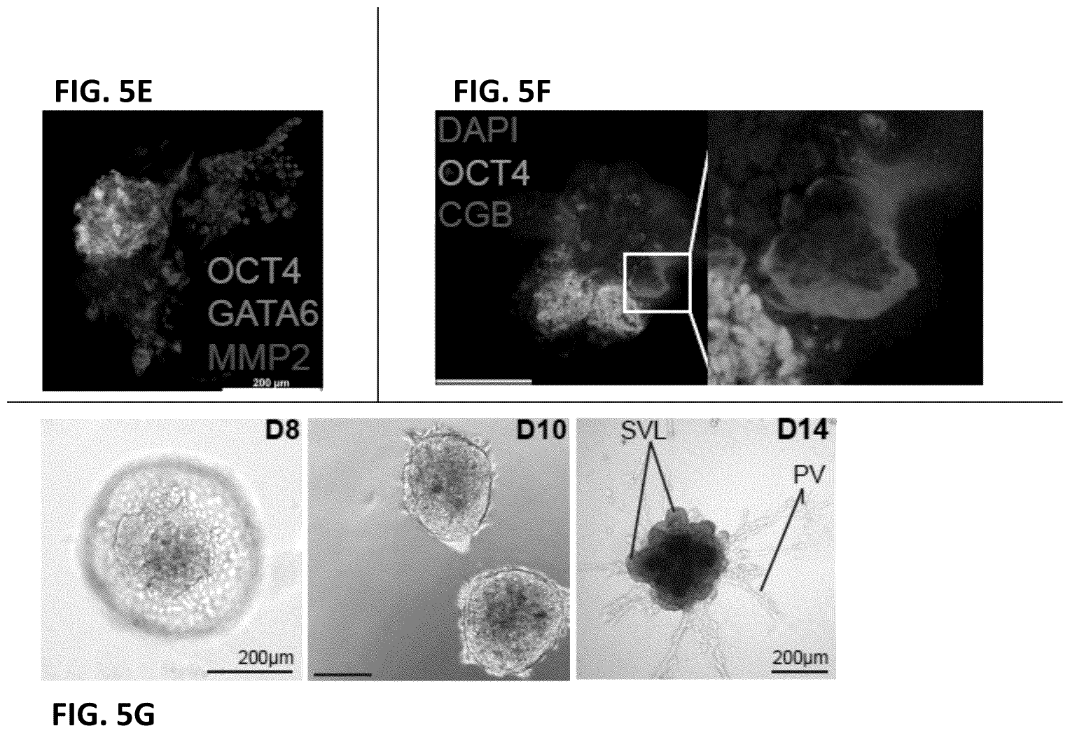

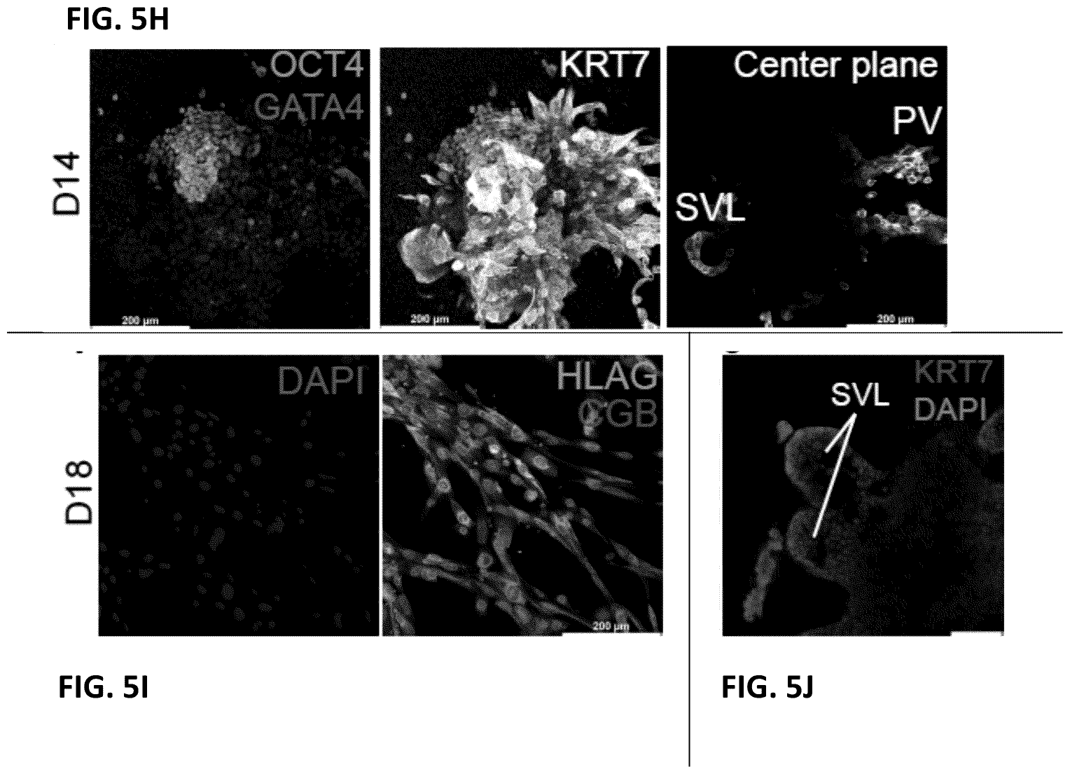

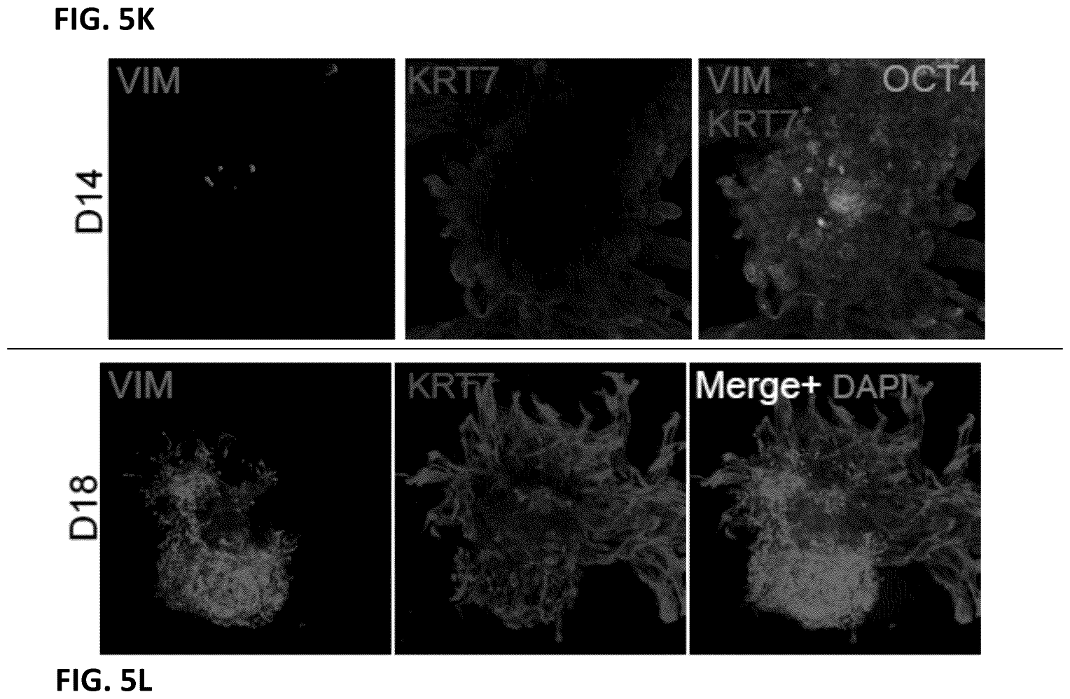

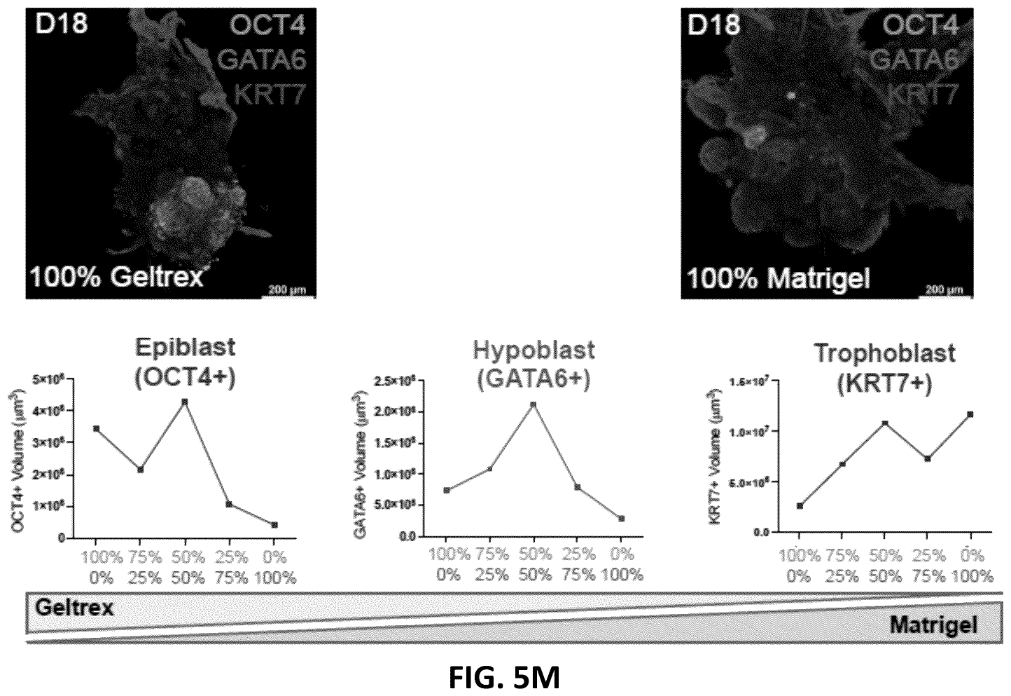

Since IVF-derived blastocysts cultured on 3D matrices capture architectural and developmental landmarks of human pre-gastrulation embryos, we considered whether a thicker ECM may be more conducive to post-implantation blastoid development. Indeed, blastoids transferred to a thick (1-2 mm) layer of Geltrex and maintained in N2B27 supplemented with estradiol (E2) maintained a more complex architecture compared to those transferred to a 2D matrix (FIG. 5B). These 3D-cultured human blastoids displayed lumen formation within the EPI compartment, which in many cases adjoined a layer of GATA4-positive PE cells, suggestive of bilaminar disc formation (FIG. 5A-C). We also observed expansion of KRT7-positive trophoblast cells, MMP2-positive invasive extravillous trophoblasts (EVT), and multinucleated hCG-positive syncytiotrophoblasts (STB) by D14 (FIG. 5D-F). Blastoids attached via the polar TE and developed two morphologically discrete trophoblast structures over a 7-day period: primary villi (PV) that co-expressed the EVT marker HLA-G and the STB marker CGB (FIG. 5G-I) and secondary villous-like (SVL) structures that were lined by KRT7 (FIG. 5G-H and FIG. 3J). Interestingly, we also observed the appearance of VIM-positive EXM cells within the EPI by D14 and their migration into the CTB compartment by D18 (FIG. 5K-L). The migration of EXM cells into the trophoblast compartment is a critical early event during morphogenesis of the placental villous tree that precedes gastrulation in the primate embryo.

We compared the impact of various commercially available matrices and noticed that blastoids transferred to a 3D matrix of Matrigel exhibited fewer GATA4-positive PE and OCT4-positive EPI cells, but more KRT7-positive trophoblast cells, compared to those on 3D Geltrex (FIG. 6B). These observations suggest that differences in the physical properties or growth factor composition of these commercially available matrices skew the balance between PE and TE lineages in post-implantation blastoids. To achieve a balanced lineage outcome, we transferred blastoids to matrices consisting of various concentrations of Geltrex and Matrigel. Blastoids cultured on a hybrid 3D matrix consisting of 50% Geltrex and 50% Matrix showed the most proportionate development of EPI, PE, and TE derivatives (FIG. 5M and FIG. 6C). Therefore, this hybrid matrix was selected to conduct single cell transcriptome profiling of extended blastoid culture at D14.

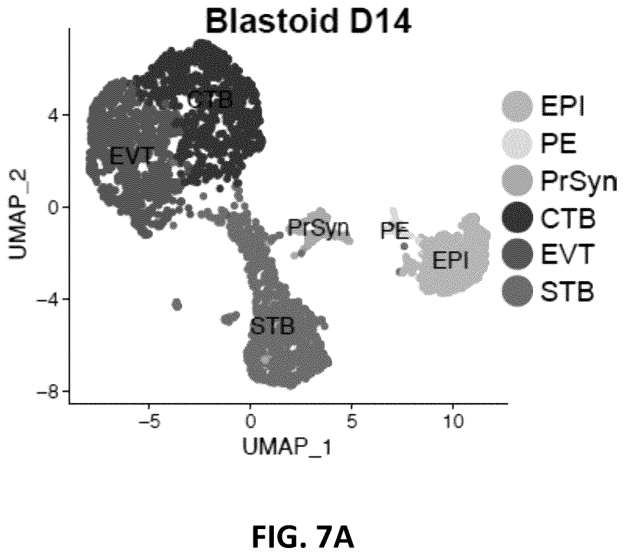

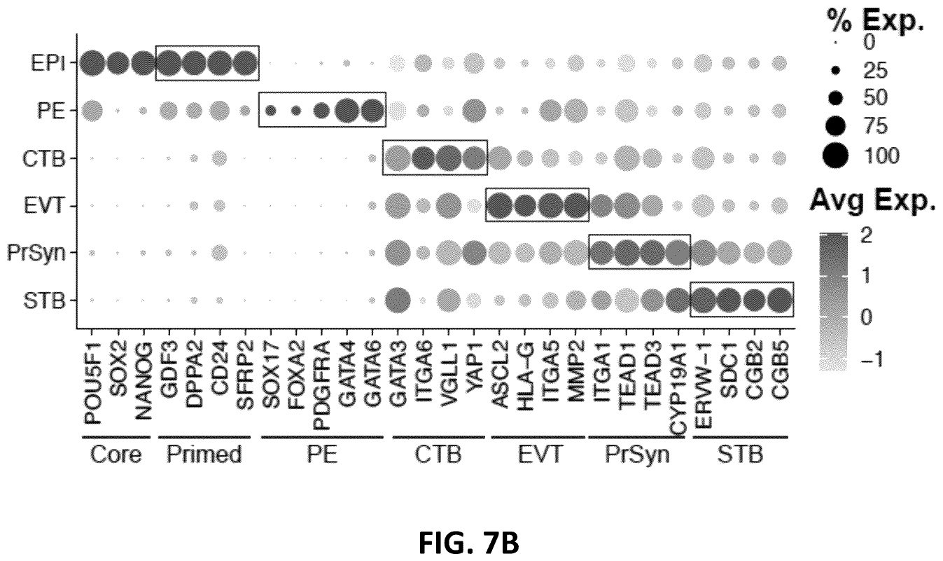

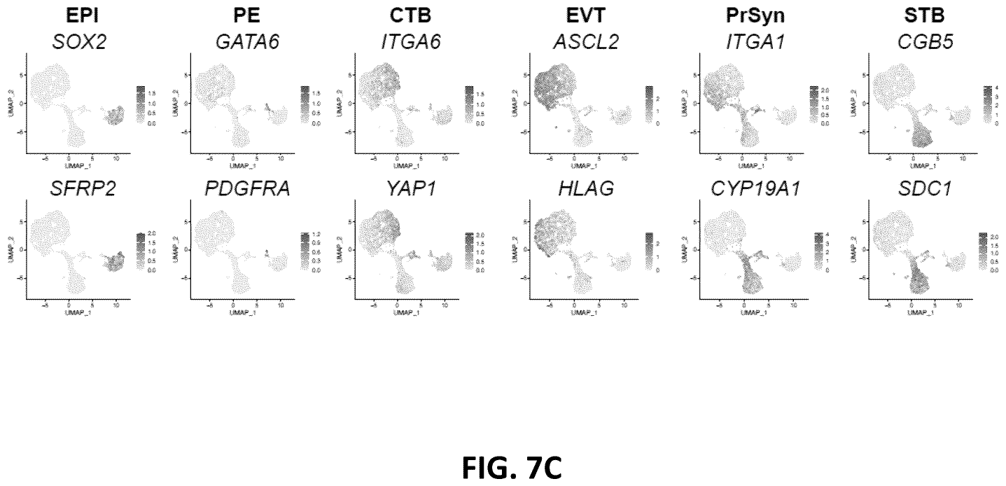

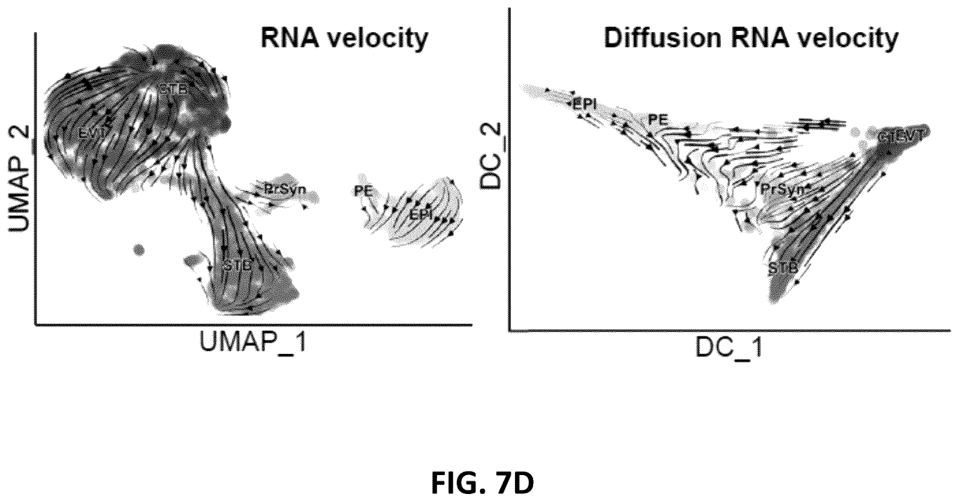

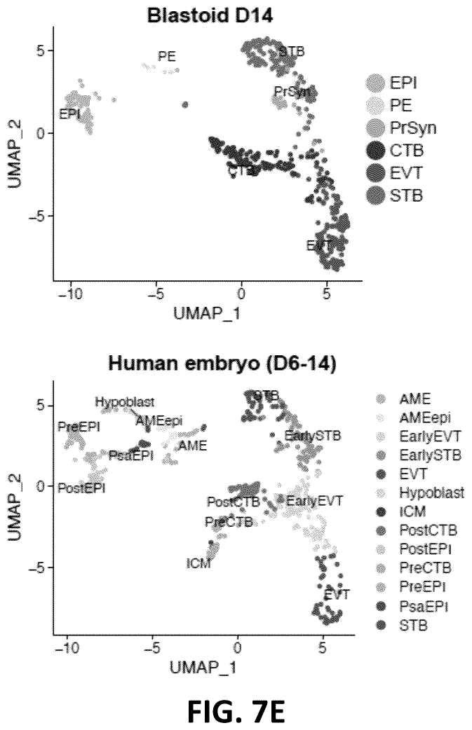

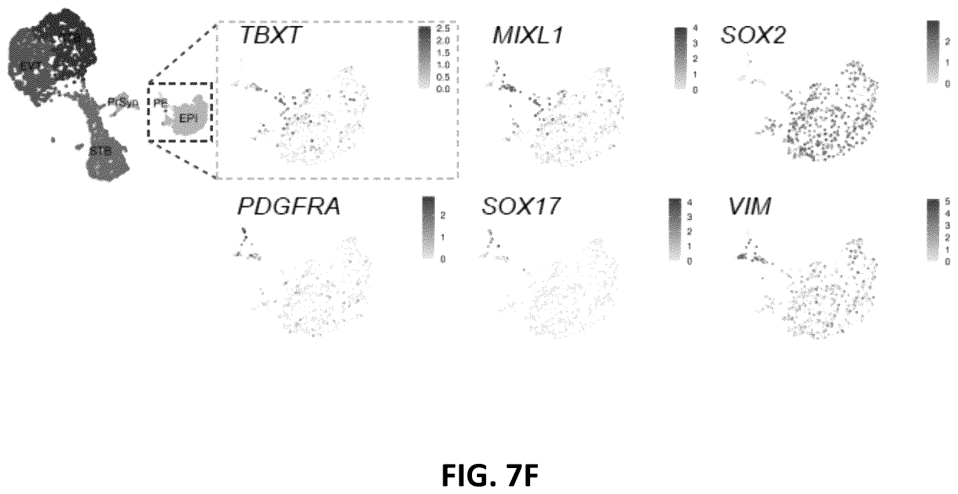

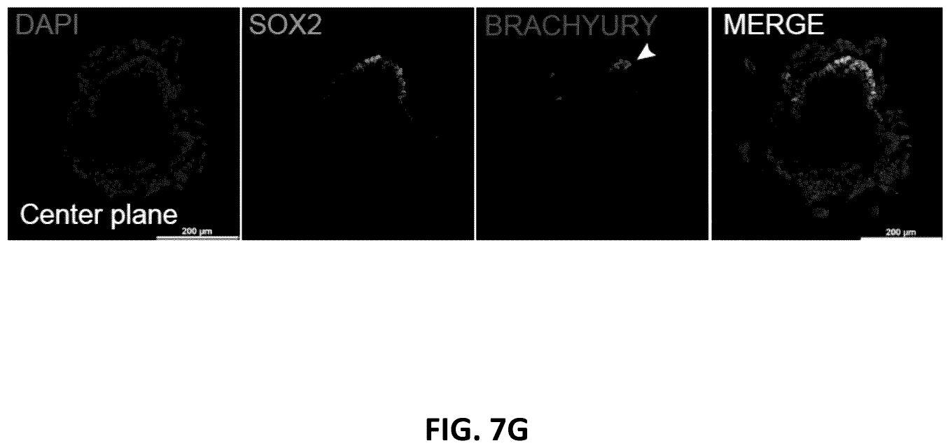

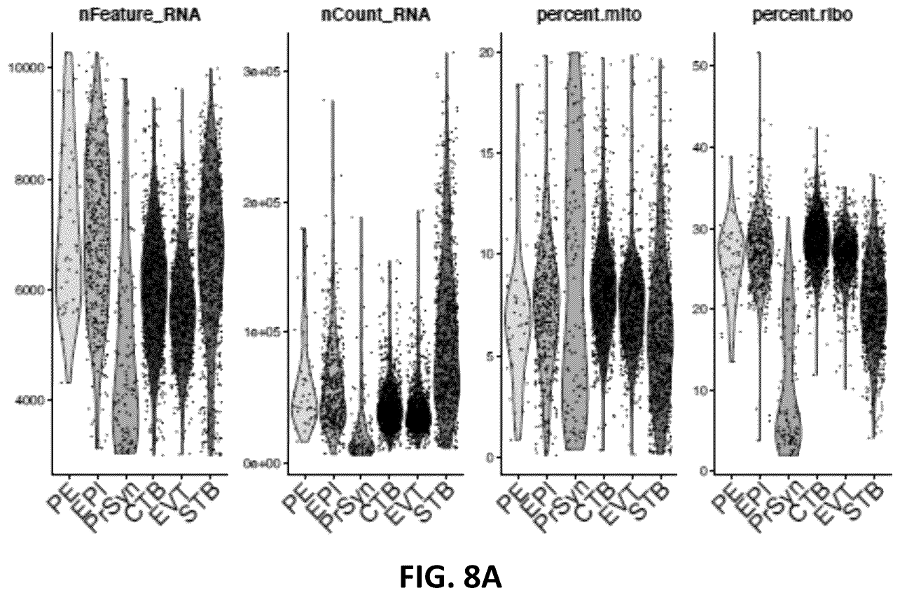

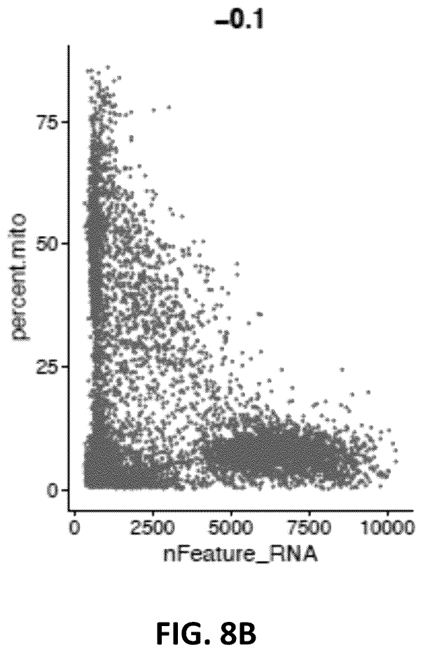

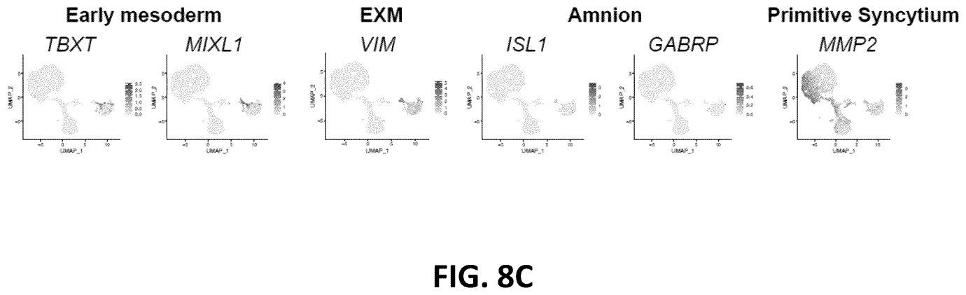

UMAP analysis revealed seven major clusters at D14, which we annotated as EPI, PE, CTB, EVT, STB, and a small cluster with mixed EVT/STB characteristics (FIG. 7A and FIG. 8A-B). The EPI cluster was marked by elevated expression of core pluripotency genes, such as POU5F1 and NANOG, but also displayed activation of markers of formative or primed pluripotency, such as CD24, DPPA2, GDF3, and SFRP2; the PE cluster was marked by GATA4, GATA6, and PDGFRA; the CTB cluster was marked by GATA3, ITGA6, VGLL1, and YAP1; the EVT cluster was marked by ASCL2, HLA-G, ITGAS, and MMP2; and the STB cluster was marked by ERVW-1, CGB2, CGB5, and SDC1 (FIG. 7B-C). The cluster with mixed EVT/STB characteristics co-expressed various EVT (ITGA1/5, MMP2) and STB (CYP19A1, ERVW-1) transcripts (FIG. 7B-C). We propose that this cluster resembles the primitive syncytium (PrSyn), which invades the maternal decidua during implantation and forms the first conduit of nutrient and gas exchange with endometrial glands. RNA velocity and diffusion map analyses revealed two prominent developmental trajectories that emerged from CTB into either EVT or STB/PrSyn fates (FIG. 70). In contrast, no trajectories were seen between embryonic and extraembryonic clusters at D14. These data suggest that, once established in the pre-implantation blastoid, the trophoblast lineage continues to autonomously generate its own specialized derivatives.

Integration with scRNA-seq data from 3D-cultured human embryos revealed close alignment of post-implantation blastoid clusters with their counterparts in the human embryo, although D14 blastoids had a smaller PE (hypoblast) cluster and lacked dedicated AME and primitive streak (PS) clusters (FIG. 7E). Upon closer examination, however, we noticed the presence of a subcluster of PS-like cells that were marked by MIXL1 and TBXT (BRACHYURY) at the intersection of the PE cluster (marked by PDGFRA and SOX17) and the EPI cluster (marked by SOX2) in D14 blastoids (FIG. 7F). IF analysis confirmed the activation of BRACHYURY within a localized region of SOX2-positive EPI cells in 3D-cultured blastoids at D18 (FIG. 7G). These data indicate that 3D-cultured blastoids are capable of activating PS markers, albeit with reduced kinetics compared to human IVF-derived blastocysts. Interestingly, VIM-positive cells were detected within the PE cluster in the single cell data of D14 blastoids (FIG. 8C), which is consistent with the postulated yolk sac origin of EXM cells in the primate embryo.

### Integrated Single Cell Profiling Reveals Key Trajectories and Driver Genes During Human Blastoid Development Up to D14

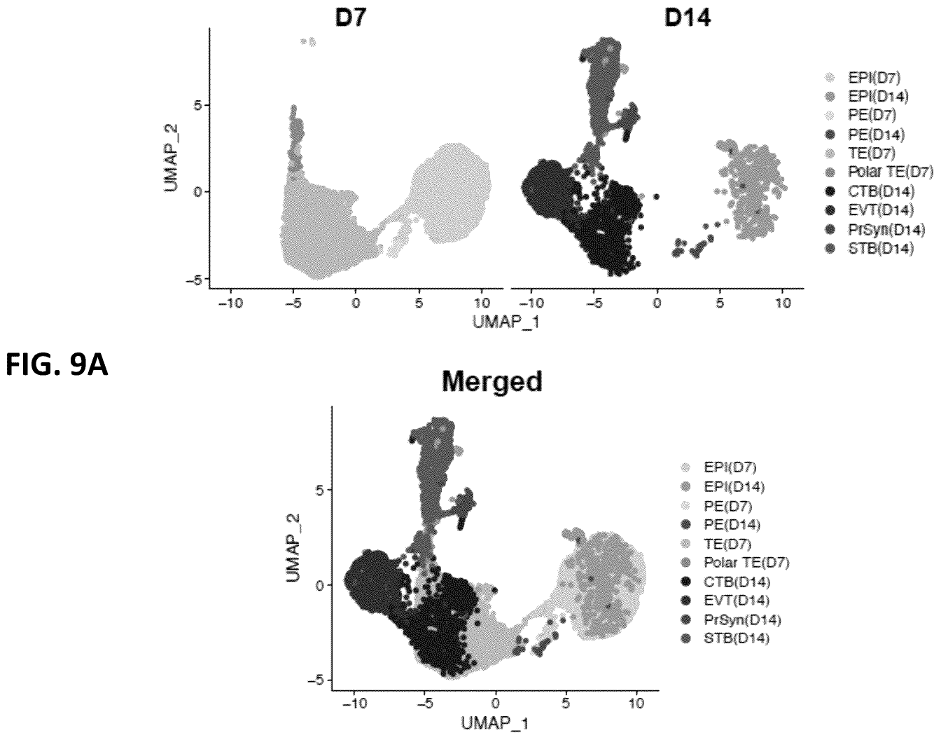

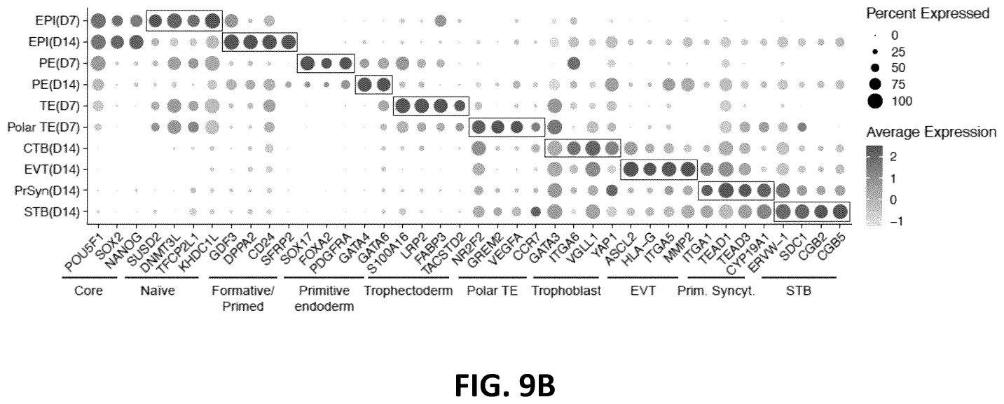

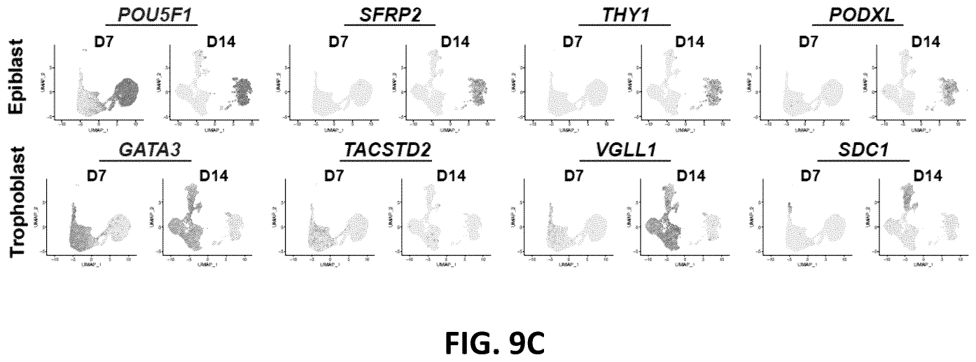

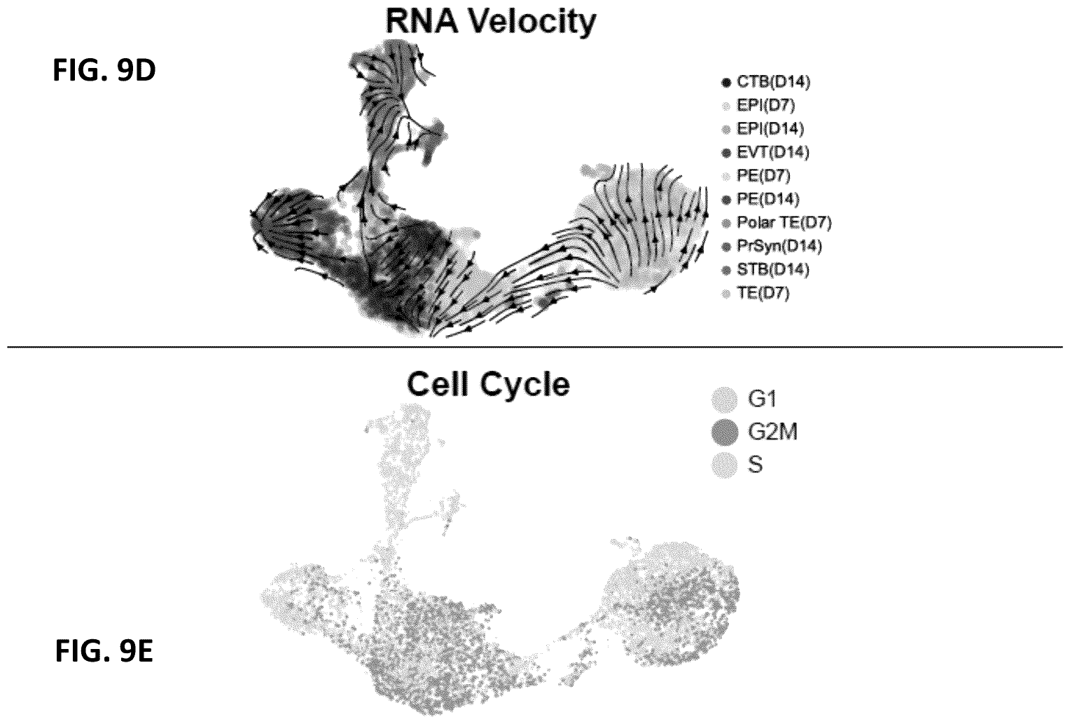

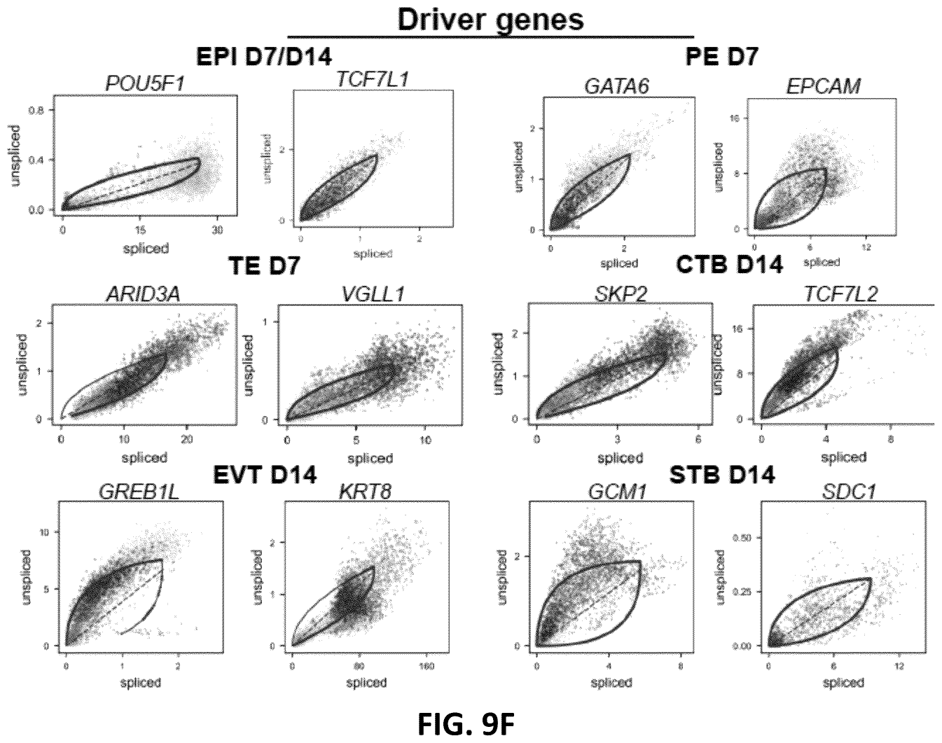

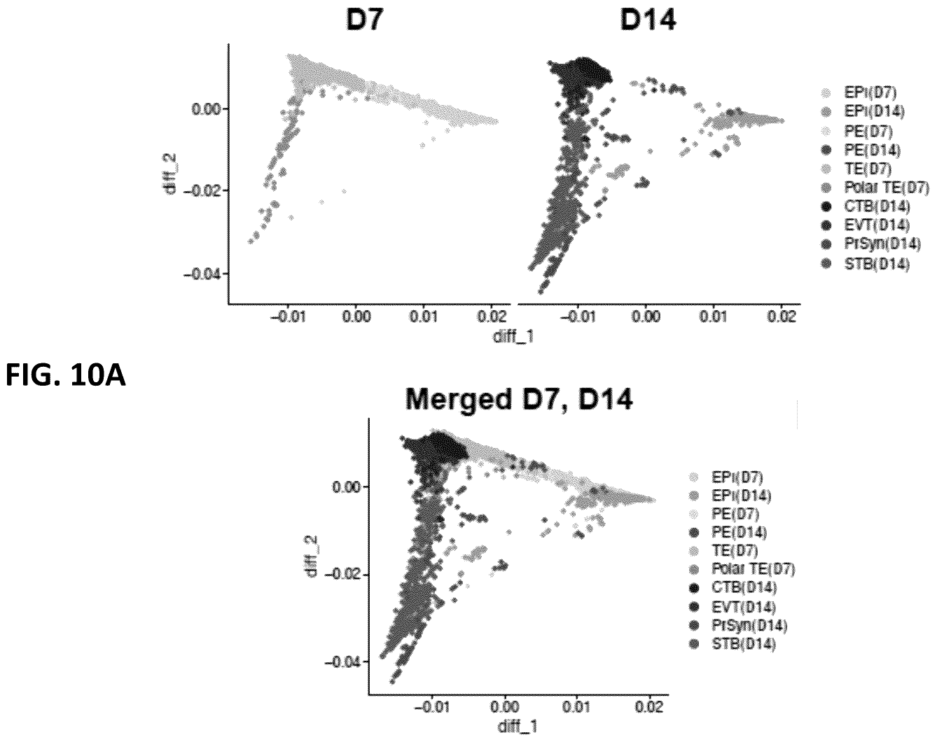

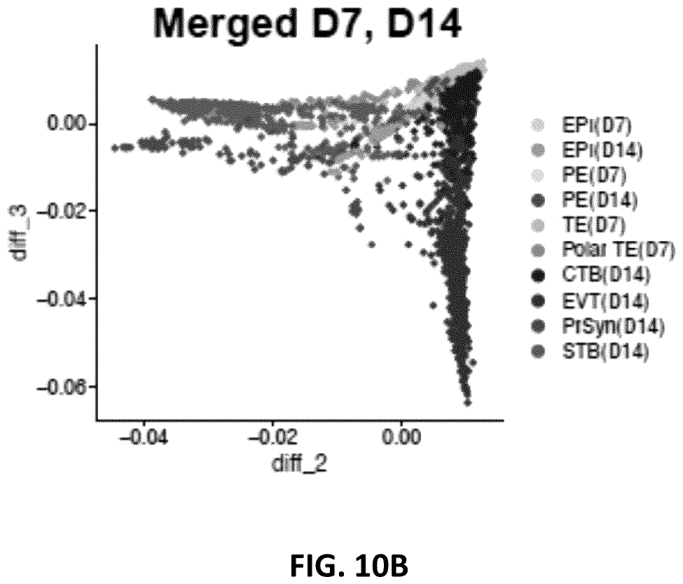

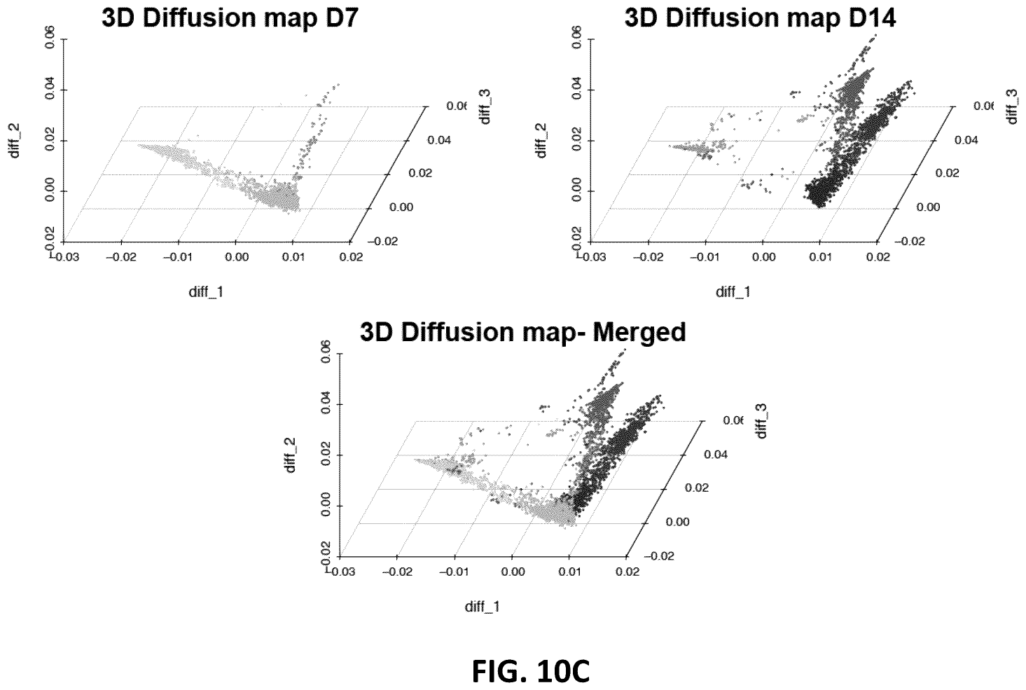

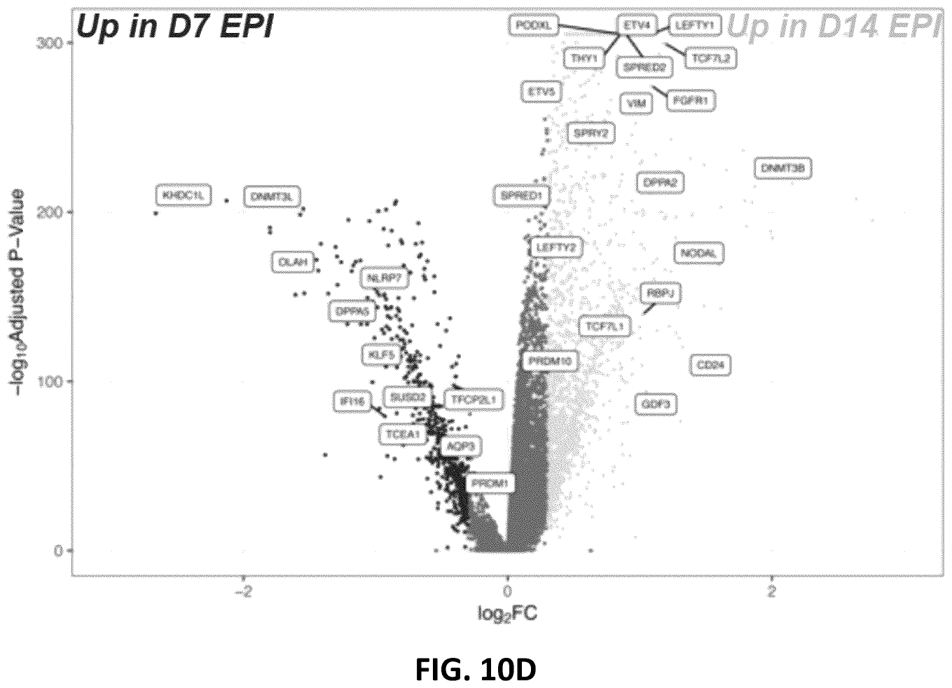

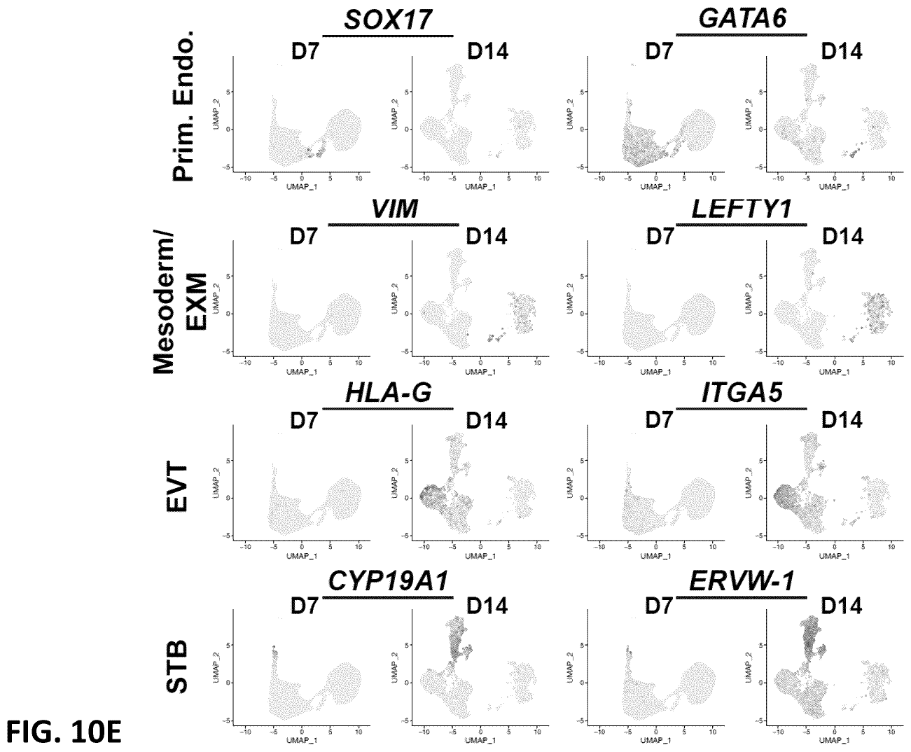

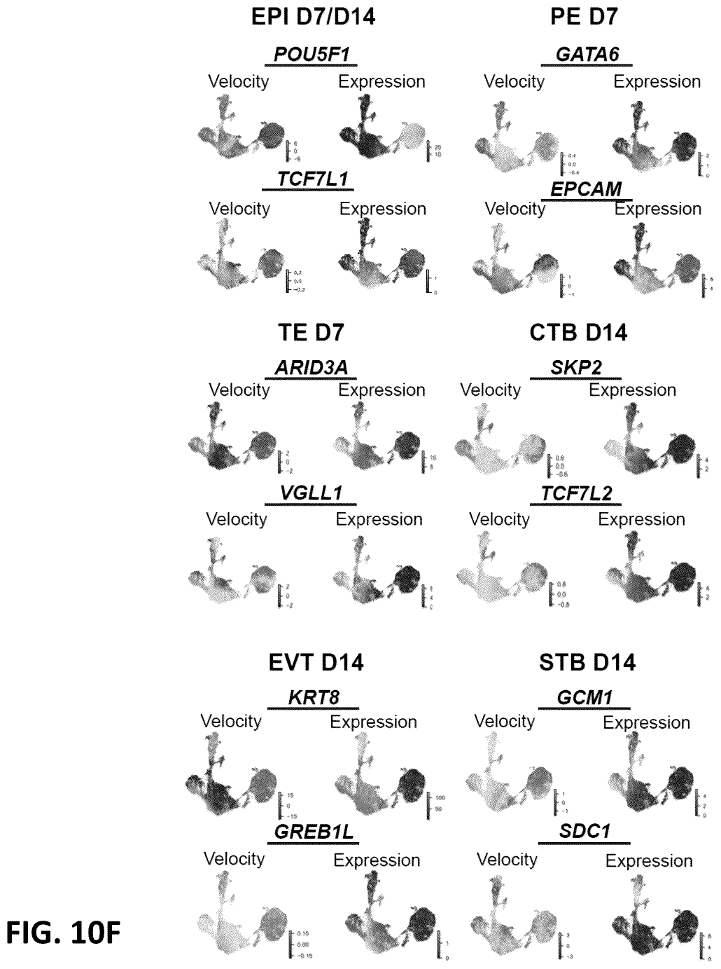

We integrated the 10× Genomics single cell gene expression profiles of D7 and D14 blastoids (FIG. 9A). Overall, EPI and TE derivatives were separated along UMAP-1, while trophoblast progenitor (TE and CTB) and differentiated cell types (EVT, STB, and PrSyn) were separated along UMAP-2. EPI subpopulations from both time points were closely aligned in this projection, as were TE (D7) and CTB (D14). In contrast, EVT, STB, and PrSyn occupied distinct positions, highlighting the diversification of trophoblast identities seen in post-implantation blastoids. Interestingly, polar TE (D7) was positioned between TE/CTB and STB/PrSyn, which suggests that these polar TE cells may give rise to syncytial hormone-producing cells during the peri-implantation window. Further support for this interpretation is provided by integrated diffusion map analysis, which suggests that polar TE precedes the formation of PrSyn and STB (FIG. 10A-C). In contrast, EVT (D14) was positioned in proximity to TE (D7) and CTB (D14) in both the UMAP and diffusion maps, suggesting that EVT differentiation proceeds via a separate trajectory that is independent of polar TE (FIG. 9A and FIG. 10A-C).

We then investigated gene expression dynamics between specific subpopulations in D7 and D14 blastoids. EPI subpopulations from both time points displayed comparable expression of core pluripotency genes, including OCT4, SOX2, and NANOG (FIG. 9B-C). Naïve-specific transcripts, such as DNMT3L, SUSD2, TFCP2L1, and KHDC1L, were downregulated in D14 EPI compared to D7 EPI, whereas primed-specific transcripts, such as PODXL, SFRP2, and THY1 were upregulated in D14 EPI (FIG. 9B-C and FIG. 10D). D7 PE showed elevated expression of SOX17, FOXA2, and PDGFRA, while D14 PE D14 showed elevated expression of GATA4 and GATA6 (FIG. 9B-C and FIG. 10E). The general trophoblast marker GATA3 was robustly expressed in D7 TE and all of its derivatives at D14 (FIG. 9C). On the other hand, S100A16, LRP2, FABP3, and TACSTD2 specifically marked D7 TE, while ITGA6, VGLL1, and YAP1 were upregulated in D14 CTB (FIG. 9C and FIG. 10E). STB-associated markers, such as CYP19A1, ERVW-1, and SDC1 first appeared in polar TE at D7, and became more widely expressed in STB at D14 (FIG. 9C and FIG. 10E). In addition, the EVT markers HLA-G and ITGAS were not expressed at D7, but showed EVT-specific upregulation in D14 blastoids (FIG. 10E).

To define cellular trajectories and predict candidate driver genes during the transition from pre-implantation (D7) to post-implantation (D14) blastoids, we applied scVelo, a method that solves the full transcriptional dynamics of splicing kinetics using a likelihood-based dynamical model. scVelo revealed three prominent trajectories leading from EPI (D7) into PE (D7), TE (D7), and EPI (D14) (FIG. 90). Additional trajectories led from TE (D7) to CTB (D14), CTB to EVT (both at D14), and polar TE (D7) to STB (D14). These observations lend further support to our prior interpretation that polar TE is the likely cell-type-of-origin for syncytial cell types in pre-villous D14 blastoids, while CTBs continue to replenish EVTs and, most likely, subsequent waves of STBs that emerge in the villous placenta. Indeed, cell cycle analysis confirmed that EPI and TE/CTB clusters are the most proliferative cell types within our blastoid dataset, while few G2/M cells were detected among the more specialized trophoblast cell types (FIG. 9E). We also used scVelo to calculate putative driver genes of these transcriptional changes based on the assumption that driver genes display pronounced dynamic behavior. This analysis identified several well-known lineage-specific regulators, such as POU5F1 (EPI), GATA6 (PE), VGLL1 (TE), SKP2 (CRB), KRT8 (EVT), and GCM1 (STB), thereby validating our approach (FIG. 9F and FIG. 10F). Interestingly, this analysis also identified TCF7L1 (formerly TCF3) and TCF7L2 (formerly TCF4) as driver genes of EPI (D14) and CTB (D14), respectively. TCF7L1 predominantly acts as a transcriptional repressor, while TCF7L2 can act either as an activator or repressor of WNT target genes. These data suggest that the WNT pathway may contribute to embryonic and extraembryonic lineage progression in our blastoid model.

### WNT Signaling Regulates Lineage Segregation During Extended Blastoid Culture

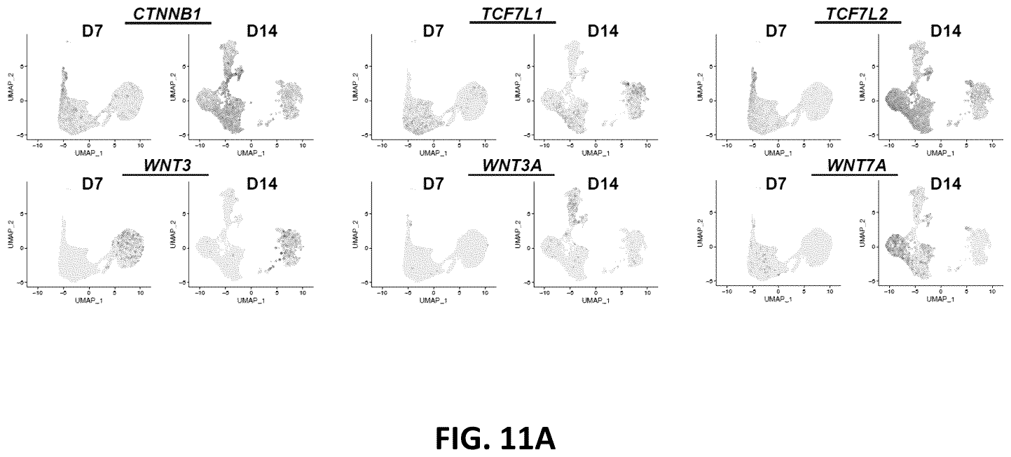

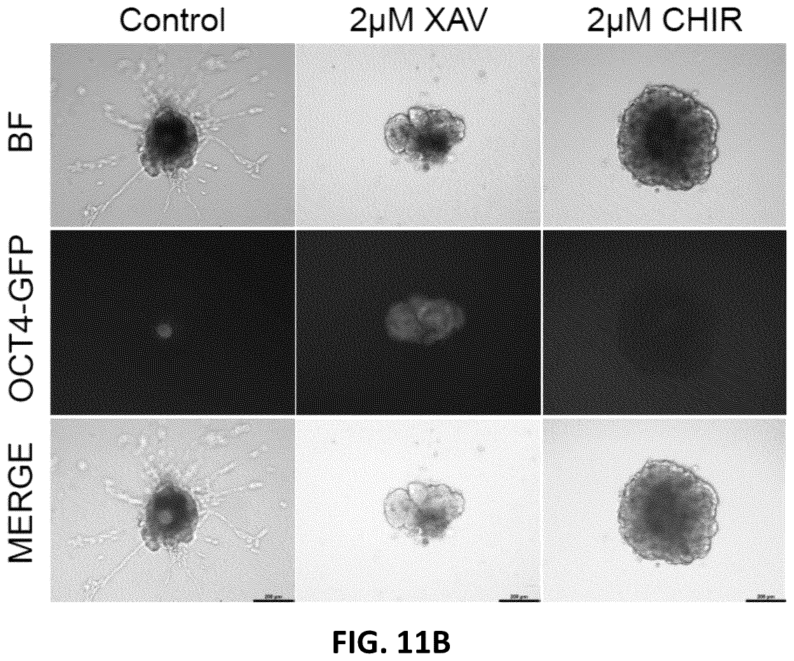

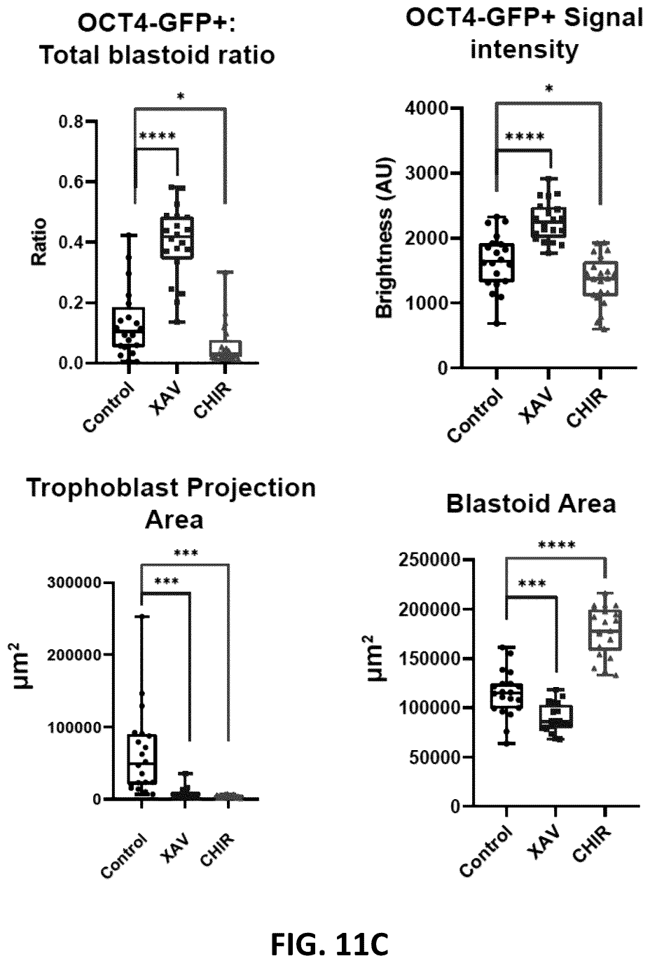

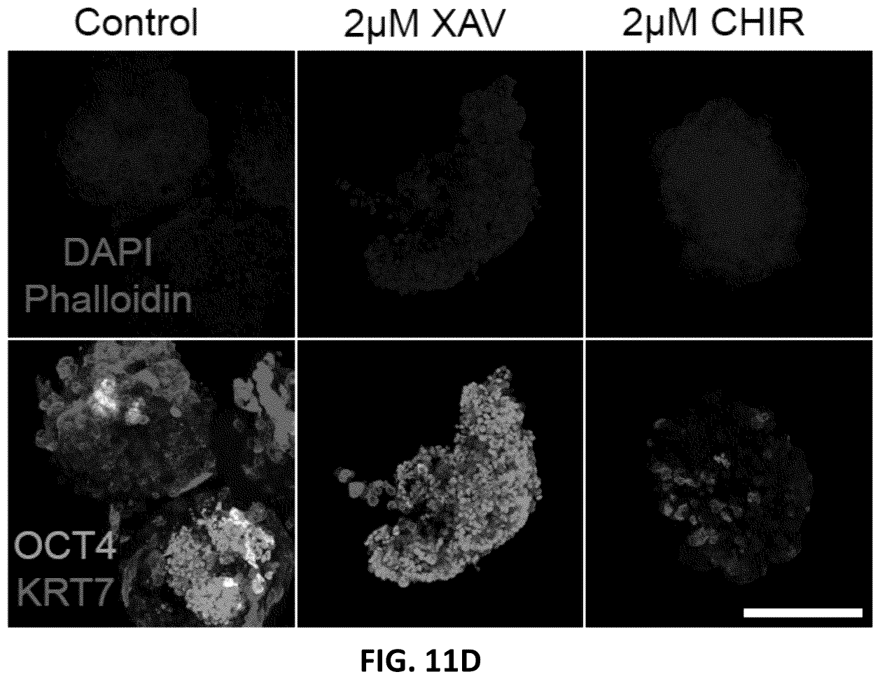

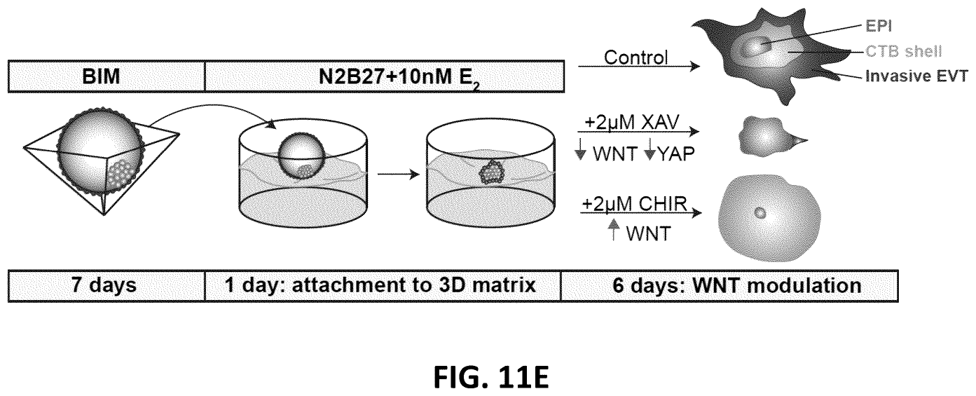

Given the identification of TCF7L1 and TCF7L2 as candidate driver genes, we sought to investigate the function of the WNT signaling pathway in lineage segregation during extended blastoid culture. WNT signaling has a well-described role in promoting axis formation and gastrulation in mice and zebrafish. Furthermore, Tcf7/1 accelerates the transition from naïve to formative pluripotency in mouse ESCs, while WNT inhibition promotes the capacitation of naïve hPSCs into a lineage-competent pluripotent state. We detected expression of several WNT ligands and pathway components in our integrated blastoid scRNA-seq dataset. In accordance with their status as driver genes, TCF7L1 and TCF7L2 were upregulated during the transition from EPI (D7) to EPI (D14) and from TE (D7) to CTB (D14), respectively (FIG. 11A). In addition, CTNNB1, the gene encoding β-catenin, was broadly expressed across embryonic and extraembryonic lineages, while specific WNT ligands were associated with EPI (WNT3), STB (WNT3A), and EVT (WNT7A) fates (FIG. 11A). These observations led us to examine the impact of modulating endogenous WNT activity on extended blastoid culture. Treatment of 3D-cultured blastoids with XAV939, a tankyrase inhibitor that negatively regulates WNT signaling as well as YAP, caused a notable expansion and increase in signal intensity of endogenous OCT4-GFP reporter activity (FIG. 11B-C). XAV939-treated blastoids also displayed significantly fewer invasive trophoblast projections. We then investigated the effect of WNT pathway stimulation by treating 3D-cultured blastoids with the GSK3 inhibitor CHIR99021. In contrast to XAV939, CHIR99021-treated blastoids exhibited a reduced ratio of OCT4-GFP+ cells and diminished intensity of OCT4-GFP reporter activity at D14 (FIG. 11B-C). These CHIR99021-treated blastoids were also larger and displayed more defined boundaries. Like XAV939-treated blastoids, the CHIR99021-treated blastoids had significantly fewer invasive trophoblast projections compared to controls. These findings were confirmed by IF staining with antibodies against KRT7 and OCT4 (FIG. 11D). We surmise that WNT inhibition promotes expansion of the EPI compartment in 3D-cultured blastoids, while the differentiation of CTBs into invasive EVTs is perturbed by either enhanced or reduced WNT pathway activity (FIG. 11E).

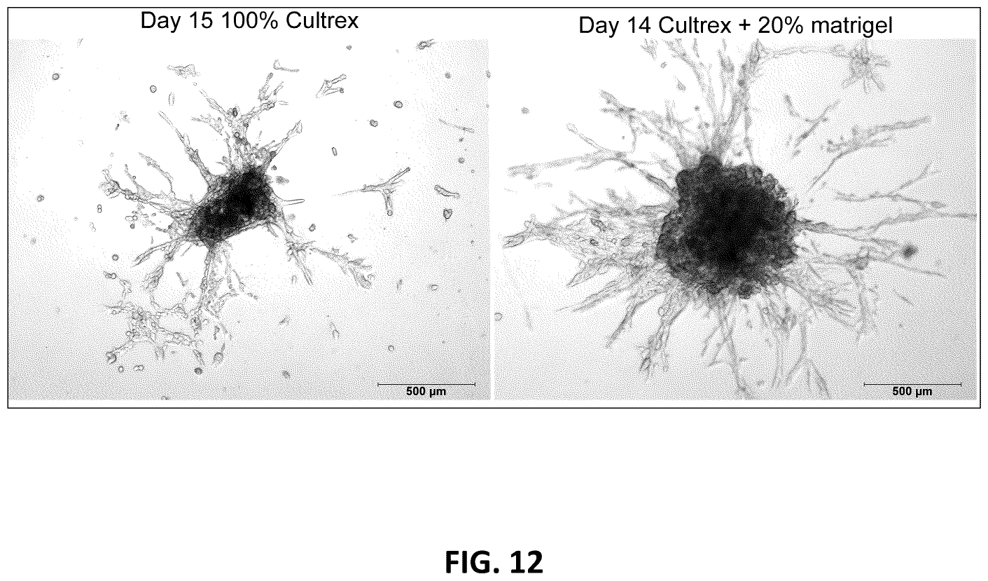

Blastoids were generated from naïve human embryonic stem cells and transferred on day 8 to 3D gels consisting of either 100% Cultrex UltriMatrix Reduced Growth Factor Basement Membrane Extract (R&D) or Cultrex+20% Reduced Growth factor hESC qualified Matrigel (Corning). Post-implantation blastoids were imaged on days 14-15. A hybrid matrix supported more robust development of embryonic and placental structures. According to the present disclosure, the use of a 3D matrix comprising 80% Cultrex Ultrimatrix Basement Membrane and 20% Matrigel supports more robust development of post-implantation blastoid structures (FIG. 12).

### Discussion

The past few years have witnessed a surge of interest in the generation of stem-cell-based embryo models. Blastoids that comprise the three major lineages of the pre-implantation embryo have been generated from naïve hPSCs, but their potential to recapitulate defining features of post-implantation development has remained uncharacterized thus far. Here we have developed an optimized protocol for blastoid generation from naïve hPSCs and characterized their capacity for extended culture on thick 3D matrices, which better mimic the physical environment of the receptive human endometrium compared to flat surfaces. These 3D-cultured blastoids capture several molecular and morphogenetic aspects of early human post-implantation development. By performing single cell analysis at two discrete stages of blastoid development (D7 and D14), we have reconstructed cellular trajectories and defined candidate driver genes of embryonic and extraembryonic lineage segregation. We also demonstrate that modulation of WNT signaling alters cell fate decisions during extended blastoid culture.

Four aspects are highlighted for further discussion: First, we have developed an optimized protocol for generating blastoids from naïve hPSCs derived in 5i/L/A. This work extends and modifies a prior study that applied sequential PE- and TE-inducing factors to generate blastoids from 5i/L/A naïve hPSCs. Compared to this prior study, we have removed Activin, CHIR99021, and FGF2 from the first two days, which indicates that exposure to minimal media devoid of inhibitors or growth factors is sufficient for naïve cell aggregation. We have also developed a simplified blastocyst induction medium (“BIM”) that enables efficient cavitation and appropriate induction of pre-implantation lineages over the next 5 days. Based on scRNA-seq analysis, EPI, PE, and TE lineages in D7 blastoids are closely aligned with their counterparts in human pre-implantation embryos, and display few, if any, off-target AME or EXM cells. We conclude that naïve hPSCs derived in 5i/L/A are capable of forming high-quality blastoids with comparable efficiency to recent studies utilizing naïve hPSCs derived in PXGL. In addition, OCT imaging revealed that the ICM specifically develops towards the bottom of the Aggrewells. These data point to a previously unknown mechanical influence on ICM development, which warrants further investigation.

Second, we have shown that human blastoids can complete the initial stages of post-implantation development when cultured on a thick 3D matrix in serum-free medium supplemented with estradiol, which mimics the receptive endometrium. This culture condition differs from prior studies that investigated blastoid attachment on tissue culture plastic or a thin ECM coating. 3D-cultured blastoids displayed several hallmarks of early post-implantation development, including lumenogenesis of the EPI compartment, rapid expansion and diversification of trophoblast lineages, and robust invasion of extravillous trophoblast cells. scRNA-seq analysis revealed a close alignment between EPI, PE, CTB, EVT, and STB lineages in 3D-cultured human blastoids and human embryos at D14. By integrating the single cell transcriptomes at D7 and D14 of blastoid development, we observed a prominent trajectory from EPI to TE at D7, but not from EPI to CTB at D14. This suggests that EPI precursors contribute to the trophoblast compartment before—but not after—implantation, in agreement with recent evidence that EPI cells in the human blastocyst can contribute to TE development. Our trajectory analysis also implicated the polar TE as a likely early source of syncytial cells during the implantation window before the CTB compartment becomes the predominant source of STBs in the villous placenta.

Third, RNA velocity analysis identified the WNT pathway effectors TCF7L1 and TCF7L2 as driver genes of EPI and CTB lineages at D14, respectively, which prompted us to evaluate the impact of WNT pathway modulation on extended blastoid culture. Treatment with a selective tankyrase inhibitor, which inhibits canonical WNT signaling, caused an expansion of the OCT4-positive domain and an increase in OCT4-GFP signal intensity within 3D-cultured blastoids. An opposite phenotype was observed using a GSK3 inhibitor, which activates canonical WNT signaling. These data suggest that WNT pathway inhibition shields the post-implantation EPI from premature differentiation, in accordance with the role of WNT inhibition in formative hPSCs. However, these inhibitors also caused significant changes in other parameters of extended blastoid culture: XAV939 reduced, while CHIR99021 increased, the size of 3D-cultured blastoids at D14 and both inhibitors caused a disappearance of invasive trophoblast projections. We postulate that XAV939 may also impair CTB development by inhibiting YAP, an important co-factor of the TEAD factors during trophoblast development. Conversely, CHIR99021 is likely detrimental to EVT differentiation, in accordance with evidence from human trophoblast organoids. Distinguishing between these multi-layered effects of WNT signaling will require cell-type-specific ablation of WNT effectors. These results illustrate how blastoids can be used to model the impact of signal perturbation on multiple embryonic and extraembryonic lineages simultaneously.

Finally, we report that human blastoids can be maintained on 3D matrices until at least D18, which resulted in the localized activation of BRACHYURY within the EPI, the migration of VIM+ EXM cells into the trophoblast compartment, and the appearance of large KRT7+ villous structures characteristic of secondary villous structures. Thus, 3D-cultured human blastoids offer an integrated model of embryonic and extraembryonic development across the implantation window, which captures the progression of the pre-implantation EPI into an early post-implantation formative state, and the differentiation of TE into post-implantation CTBs and specialized trophoblast derivatives. The current disclosure opens the door to defining the genetic and epigenetic mechanisms governing a previously inaccessible stage of human embryogenesis, and how these mechanisms may be perturbed during implantation failure and early pregnancy loss. Our study may also provide a steppingstone for generating more advanced human embryo models that are capable of undergoing gastrulation. This will likely require culture modifications to enhance the specification and/or proliferation of the amnion and yolk sac, which were underrepresented in 3D-cultured blastoids at D14, but serve as key signaling centers during gastrulation.

### STAR Methods

### Key Resources Table

| | | |
|---|---|---|
| REAGENT or RESOURCE | SOURCE | IDENTIFIER |

Antibodies

| | | |
|---|---|---|
| anti-KRT7 | Dako | M7018 |
| anti-MMP2 | Cell Signaling | 40994 |
| anti-GATA3 | Cell Signaling | 5852S |
| anti-GATA6 | R&D | AF1700 |
| anti-SOX2 | Cell Signaling | L1D6A2 |
| anti-SOX17 | R&D | AF1924 |
| anti-hCG beta | Abcam | ab53087 |
| anti-MMP2 | Cell Signaling | 40994S |
| anti-OCT4 | Cell Signaling | 75463 |
| anti-OCT4 | Cell Signaling | 2750S |
| anti-HLAG | Santa Cruz | sc-21799 |
| anti-GATA4 | Invitrogen | 14-9980-82 |
| anti-BRACHYURY/T | Cell Signaling | 81694 |
| anti-Vimentin | Cell Signaling | 5741 |
| anti-IFI16 | NovusBio | NBP1-83118 |
| anti-NR2F2 | Perseus | PP-H7/147-00 |
| | Proteomics | |
| anti-TEAD1 | Cell Signaling | 12292 |
| anti-YAP | Cell Signaling | 14074 |
| anti-ZO-1 | Invitrogen | 33-9100 |
| Secondary ab- Donkey anti-mouse 488 | Invitrogen | A21202 |
| Secondary ab- Donkey anti-rabbit 488 | Invitrogen | A21206 |
| Secondary ab- Donkey anti-mouse 555 | Invitrogen | A31570 |
| Secondary ab- Donkey anti-rabbit 555 | Invitrogen | A31572 |
| Secondary ab- Donkey anti-goat 647 | Invitrogen | A32849 |

Chemicals, peptides, and recombinant proteins

| | | |
|---|---|---|
| Phalloidin-670 | Cytoskeleton | PHDN1 |
| Phalloidin-405 | Abcam | AB176752 |
| A83-01 | Peprotech | 9094360 |
| SB431542 | BioVision | 1674 |
| Valproic Acid | Sigma-Aldrich | P4543 |
| Insulin-Transferrin-Selenium- | Gibco | 51500056 |
| Ethanolamine (ITS -X) | | |
| L-Ascorbic acid | FujiFilm | 013-12061 |
| CHIR99021 | R&D | 4423 |
| Y-27632 | Stemgent | 04-0012 |
| PD0325901 | Stemgent | 04-0006 |
| IM-12 | Enzo | BML-WN102 |
| SB590885 | Tocris | 2650 |
| WH-4-023 | A Chemtek | S1180 |
| rhEGF | Peprotech | AF-100-15 |
| rhR-Spondin1 | Peprotech | 120-38 |
| rhFGF2 | Peprotech | 100-18B |
| rhNRG1 | Cell Signaling | 5218SC |
| rhBMP4 | R&D Systems | 314-BP |
| Activin A | PeproTech | 120-14 |
| LIF | PeproTech | 300-05 |
| StemPro EZPassage | Invitrogen | 23181-010 |
| hESC qualified Matrigel | Corning | 354277 |
| Dispase | Stem Cell | 07923 |
| | Technologies | |
| TrypLE Express | Gibco | 12604-013 |
| Primocin | Invitrogen | ant-pm-1 |
| N-Acetyl-L-Cysteine | Sigma Aldrich | A9165 |
| Knock-out Serum Replacement | ThermoFisher | 10828028 |
| Collagen IV | Corning | 354233 |
| Papain | MP Biomedicals | 100921 |
| N-2 Supplement | Gibco | 17502-048 |
| B-27 Supplement | Gibco | 17504-044 |
| Aggrewell 400 24 well plates | Stemcell | 34415 |
| | Technologies | |
| Ibidi micro well chamber slides | Ibidi | 80826 |
| 16% Paraformaldehyde | Electron | 15710 |
| | Microscopy | |
| | Sciences | |
| beta-Estradiol | Sigma-Aldrich | E8875 |

Critical commercial assays

| | | |
|---|---|---|
| TaqMan Fast Advanced Master Mix | Applied | 4444556 |
| | Biosystems | |
| DNeasy Blood and Tissue Kit | Qiagen | 69504 |
| MMP2 ELISA Kit | Abcam | ab100606 |
| hCG ELISA Kit | CalBiotech | HC251F |

Deposited data

| | | |
|---|---|---|
| Raw and Processed data | Present disclosure | GEO: |
| | | GSE172241 |
| A developmental landscape of 3D- | Xiang et al., 2020 | GSE136447 |
| cultured human pre-gastrulation embryos | | |
| Single-Cell RNA-Seq Reveals Lineage | Petropoulos et al., | ArrayExpress: |
| and X Chromosome Dynamics in Human | 2016 | E-MTAB-3929 |
| Preimplantation Embryos | | |

Experimental models: Cell lines

| | | |
|---|---|---|
| H9 (hESC) | WashU GEiC | N/A |
| H1 (hESC) | WashU GEiC | N/A |
| WIBR3 (hESC) | Whitehead | N/A |
| | Institute | |
| WIBR3 OCT4-GFP (hESC) | Whitehead | N/A |
| | Institute | |
| WIBR1 (hESC) | Whitehead | N/A |
| | Institute | |
| RUES2-GLR (hESC) | Solnika-Krezel OR | N/A |
| | Brivanlou | |

Software and algorithms

| | | |
|---|---|---|
| FlowJo_v10.6.2 | FlowJo ™ | RRID: SCR_0085 |
| | | 20; flowjo |
| Prism 9 | GraphPad | RRID: SCR_0027 |
| | | 98; graphpad |
| FIJI | NIH | imagej (Fiji) |
| Amira | ThermoFisher | N/A |
| R 4.0.0 | R project | N/A; r-project |
| Cellranger 5.0.0 | 10Xgenomics | support.10xgenomics |
| | | (single-cell-gene- |
| | | expression/ |
| | | software/ |
| | | downloads/latest) |
| ClusterProfiler 4.0.0 | Bioconductor | bioconductor |
| | | (packages/ |
| | | release/bioc/ |
| | | clusterProfiler) |
| Seurat 4.0 | Satija Lab | satijalab (Seurat) |
| pheatmap 1.0.12 | CRAN | cran.r-project |
| | | (web/packages/ |
| | | pheatmap/index) |

### Experimental Model and Subject Details

### Cell Lines and Culture Conditions

Primed human embryonic stem cells (H-9, H1, WIBR1, WIBR3, WIBR3 OCT4-GFP, and RUES2-GLR) were maintained in mTeSR1+(StemCell Technologies 100-0276) with hESC-qualified Matrigel (Corning 354277) coated plates at 37° C., 5% C02, and 100% humidity. Primed cells were passaged with Dispase (StemCell Technologies 07923) and dissociated into small squares with the StemPro EZPassage stem cell passaging tool (Thermo Fisher Scientific 23181-010) for general maintenance.

Naïve conversion of primed PSCs was performed with 5i/L/A. Primed hPSCs were dissociated into single cells with Accutase (Stem Pro A11105-01) and 250,000 cells were plated upon a bed of mitomycin C inactivated mouse embryonic fibroblasts (iMEFs) within 6 well dishes and cultured for two days in mTeSR1+ with 10 uM ROCK inhibitor (Y-27632 Stemgent #04-0012). Following two days in mTeSR1+10 μM Y-27632, the medium was changed to 5i/L/A medium [1:1 DMEM/F12 (Gibco, 11320), 1:1 Neurobasal (Gibco, 21103), 1× N2 100× supplement (Gibco, 17502), 1× B27 50× supplement (Gibco, 17504), 1× GlutaMAX, 1×MEM NEAA (Gibco, 11140), 0.1 mM β-mercaptoethanol (Millipore Sigma, M3148), 1% penicillin-streptomycin (Gibco 15140-122), 50 μg/ml BSA Fraction V (Gibco, 15260), and the following small molecules and cytokines: 1 μM PD0325901 (Stemgent, 04-0006), 1 μM IM-12 (Enzo, BML-WN102), 0.5 μM SB590885 (Tocris, 2650), 1 μM WH4-023 (A Chemtek, H620061), 10 μM Y-27632 (Stemgent, 04-0012), 20 ng/mL recombinant human LIF (PeproTech, 300-05), and 10 ng/mL Activin A (Peprotech, 120-14)]. Naïve stem cells are maintained at 37° C., 5% CO₂, 5% O₂, and 100% humidity. Medium was changed every 1-2 days until domed shaped colonies emerged and begin proliferating (approximately 10-12 days). Naïve stem cells were passaged with Accutase for 5 minutes at 37° C. The Accutase and cell mixture was inactivated in fibroblast medium [DMEM (Millipore Sigma, #SLM-021-B) supplemented with 10% FBS (Cytiva, SH30088.03), 1× GlutaMAX (Gibco, 35050), and 1% penicillin-streptomycin (Gibco, 15140)] and centrifuged 1000 rpm, 5 minutes. The cells were washed once in DMEM/F12 and centrifuged again at 1000 rpm, 5 minutes. The cells were resuspended in 5i/L/A medium and passaged onto pre-made iMEF-coated plates.

### Generation of Blastoids

Naïve hPSCs were dissociated with Accutase for 5 minutes at 37° C. Single cell dissociate naïve stem cells and inactivate accutase enzymes with fibroblast medium. Centrifuge this suspension 1000 rpm for 5 minutes. Remove supernatant and wash once with DMEM/F12, centrifuge 1000 rpm for 5 minutes. Aspirate supernatant and resuspend in 2 mL of 5i/L/A medium. Remove iMEFs from the cell suspension by incubation on gelatin-coated plates for 30-45 minutes at 37° C. 5% CO₂. Collect cells and count cells to plate 100,000 cells per 24 well Aggrewell (StemCell Technologies) well. Coat the aggrewells with Anti-adherence solution (StemCell Technologies) for 10 minutes at room temperature. Wash wells once with DMEM/F12, then add 1 mL of 5i/L/A medium. Add naïve stem cells to Aggrewells, 100,000 cells per 500 μL, total medium should be 1.5 mL per well. Centrifuge the plate 1000 rpm for 2 minutes for equal distribution of cell number per microwell. Incubate at 37° C. 5% CO₂ and 5% 02 for one day. The following day, change medium to N2B27 without antibiotics [1:1 DMEM/F12:Neurobasal (Gibco), 1×N2 100× supplement (Gibco, 17502), 1×B27 50× supplement (Gibco, 17504), 1× GlutaMAX (Gibco 35050), 1×MEM NEAA (Gibco, 11140), 0.1 mM β-mercaptoethanol (Millipore Sigma, 8.05740), 1 mM Na Pyruvate (Corning 25-000 Cl)]. Change 1 mL from each well twice to wash out 5i/L/A medium. Change medium again the following day with the same N2B27 media without antibiotics. On the third day, exchange N2B27 medium with blastoid induction medium (BIM) [1:1 DMEM/F12: Neurobasal, 0.5×N2 100× supplement (Gibco, 17502), 0.5×B27 50× supplement (Gibco, 17504), 0.5× GlutaMAX, 0.5×MEM NEAA (Gibco, 11140), 1 mM Na Pyruvate (Corning 25-000 Cl), 0.1 mM β-mercaptoethanol (Millipore Sigma, M3148), 0.5% ITS-X (Gibco 51500-056), 0.5% Knock-out Serum Replacement (Gibco 10828028), 0.1% FBS (Cytiva, SH30088.03), 1 μM PD0325901 (Stemgent 04-0006), 1 μM A83-01 (Peprotech 9094360), 0.5 μM WH-4-023 (A Chemtek S1180), 0.25 μM IM-12 (Enzo BML-WN102), 25 ng/mL rhEGF (Peprotech AF-100-15), 3 μg/mL Ascorbic Acid (FujiFilm 013-12061), and 400 μg/mL Valproic acid (Sigma Aldrich P4543)]. Wash this medium twice, 1 mL each wash, to replace the medium on this day. Each day following, change medium once. Depending on pure domed morphology and proliferation rate of the starting naïve cells, cavitation should start to occur on day 6 and occur by day 7 or 8. Some cell lines require one additional day. Remove cavitated blastoids from Aggrewells with a rounded (not sharp) glass pipet, move them to a 10 cm dish to pick out blastoids with good morphology for further experimentation. All blastoids were generated from 5i/LA naïve conditions and used within 10 passages.

### Blastoid Post-Implantation Assays

Blastoids can be transferred on either day 7 or day 8 of growth. Due to the inaccessibility of commercially available extracellular matrix products during the experimentation phases of this project, multiple matrices were utilized in this study-hESC qualified growth factor-reduced Matrigel (Corning), hESC qualified growth factor reduced Geltrex (Gibco), and Cultrex UltriMatrix reduced growth factor basement membrane extract (R&D, BME001). Matrigel favored the outgrowth of the trophoblast lineage, while a mixture of Matrigel and Geltrex resulted in a balance of trophoblast and embryonic lineages. Geltrex was however highly inconsistent lot to lot, and so Cultrex Ultrimatrix was found to be more reliable and produced similar results as Geltrex. Matrices were thawed on ice and kept cold until crosslinking was desired. Matrices mixed together (Matrigel/geltrex) were mixed following thawing and plated into ibidi microwells (120 μL/well) (ibidi, 80826) or alternatively 96 well plates (40 μL/well). Matrices were solidified at 37° C. for 30 minutes. Wash matrices 2 times with DMEM/F12 before adding blastoids. Add N2B27+10 nM estradiol without antibiotics to each well. Plate desired numbers of blastoids to each well (3-5 in an ibidi microwell, 1-2 in a 96 well plate). Place the plate in the incubator (37° C., 5% CO₂, 5% 02) for 24 hours. Replace media daily with N2B27+10 nM estradiol without antibiotics. If blastoids are added at 7 days, the following day they may be loosely attached. Wait approximately 28-30 hours following seeding to ensure blastoids are well anchored before changing medium. Polar TE side attaches and the ICM expands compared to the day before when properly attached. On the following day (day 9) the TE collapses. Migratory EVT emerge at day 13. Maintaining proper pH is important for blastoids post implantation, the medium should be at pH=7.2-7.4 by pre-equilibration in the incubator or use freshly made N2B27 that has not oxidized too excessively (media should be pink/orange in color, never purple).

### Optical Coherence Tomography

The images were acquired by a high-resolution spectral-domain Optical Coherence Tomography (OCT). The system incorporates a broadband light source (center wavelength: 846 nm, bandwidth: 168 nm, SUPERLUM, cBLMD-T-850-HP), a spectrometer as the detector (Max line rate: 80 kHz, Wasatch Photonics, CS800-840/180-80-OC2K-U3) and the sample and reference arms to form the Michelson interferometry. The beam splitter evenly divides the source beam into the sample and reference arm. An XY Galvo scanner (Thorlabs, GVS102) allows 2D scanning across the plane. With the 10× objective lens, the system can achieve a lateral resolution of 2.19 and an axial resolution of 1.6 μm. The field of view of each image was about 3 mm×3 mm. The imaging was performed from Day 1 to Day 6 post seeding day. Each image comprises 1000 A-scans per B-scan and 1000 B-scans. The three-dimensional OCT images were reconstructed by Amira, and the further analysis such as size measurements were performed using ImageJ. 3D rendering was created using Imaris software.

### Immunofluorescence

Blastoids at day 7-8 were manually separated with a glass pipet and placed in 4% paraformaldehyde for 20 minutes at room temperature. Wash blastoids 3 times with PBS+0.05% BSA. Permeabilize and block in PBS+4% BSA+5% FBS+0.5% Triton-X overnight at 4° C. Replace blocking and permeabilization solution with antibodies diluted in staining solution (PBS+4% BSA+5% FBS+0.01% Tween-20). Incubate overnight at 4° C. Wash 3 times in PBS+0.05% BSA solution. Resuspend blastoids in staining solution with diluted secondary antibodies (1:300) and DAPI (1:1000) or Phalloidin (200 ng/mL). Incubate overnight at 4° C. Wash 3 times in PBS+0.05% BSA. Mount blastoids between two coverslips and a Grace biolabs silicon isolator (Grace Bio Labs 664570) in PBS+0.05% BSA. Blastoids were imaged with a Leica SP-8 confocal microscope or a Leica Thunder Live Cell Compound Microscope and images were processed in LASX software.

Post implantation blastoids in 2D were fixed for 15 minutes at room temperature in 4% paraformaldehyde. Staining procedure was 1 hour in blocking and permeabilization solution (PBS+4% BSA+5% FBS+0.5% Triton-X) followed by diluted primary antibodies in staining solution (PBS+4% BSA+5% FBS+0.01% tween 20) over night at 4° C. 2D samples were washed 3 times in PBS, 5 minutes per wash. Secondary antibodies (1:300) and DAPI (1:1000) were diluted in staining solution and allowed to bind at room temperature for 2 hours. Wash 3 times with PBS and mount in ProLong Gold antifade mounting reagent (Invitrogen P36930)

3D structures were removed from 3D ECM with a large-bore glass pipet. ECM was removed with Cell Recovery Solution (Corning 354253) on ice for 30 minutes. 3D post implantation blastoids were fixed in 4% PFA for 30 minutes at room temperature. Wash 3 times in PBS+0.05% BSA, 10 minutes for each wash. Block and permeabilize post implantation blastoids in PBS+4% BSA+5% FBS+0.5% Triton-X overnight at 4° C. Replace permeabilization solution with staining solution (PBS+4% BSA+5% FBS+0.01% Tween 20) and diluted primary antibodies. Incubate overnight at 4° C. Wash 3 times in PBS+0.05% BSA, 10 minutes in each wash. Add diluted secondary antibodies (1:300) and DAPI (1:1000) or Phalloidin (follow product dilution recommendation) in staining solution. Incubate overnight at 4° C. Wash 3 times in PBS+0.05% BSA 10 minutes in each wash. Mount in clearing solution (60% Glycerol, 2.5M Fructose) between two coverslips and a Grace Bio Labs Silicone isolator (Grace Bio Labs 664570). 3D post implantation blastoids were imaged with a Leica SP-8 confocal microscope and images were processed in LASX software.

Signal volumes were measured in Imaris software. Total numbers of blastoids measured was 2-3 blastoids per Geltrex-Matrigel combination. Graphs were generated in GraphPad Prism.

### Post Implantation WNT Modulation

WIBR3 OCT4-GFP blastoids were grown to 7 days in blastoid generating conditions (see above) and blastoids with localized single ICM, continuous trophectoderm, and with a round blastocoel cavity were chosen by hand and plated upon a pre-formed 3D matrix (Cultrex UltriMatrix) formed with a 96 well plate (40 μL of Cultrex per well). 2 blastoids per well were plated among 10 wells for each condition in N2B27+1 mM Na Pyruvate+10 nM Estradiol (E2). Blastoids were allowed to attach for approximately 24-30 hours in the incubator (5% CO₂, 5% O₂, 37° C., 100% humidity). When blastoids attached, TE collapsed, and ICM expanded, medium was changed to N2B27+1 mM Na Pyruvate+10 nM E2+2 μM XAV939 or +2 μM CHIR99021. Media was changed daily and grown in a 37° C., 5% CO₂, 5% O₂ incubator. Blastoids were fixed in 4% paraformaldehyde as described above on day 14 of total blastoid growth.

### Method Details

### Quantification and Statistical Analysis

### ELISA Data Analysis

Graphpad Prism was used to create visual graphs and analyses. Statistical significance was determined by a one-way ANOVA with Sidak's multiple comparisons test. Significance was determined by p<0.05. *P value<0.05, **P value<0.01.

### Blastoid Cavitation Rate and Blastoid Size Quantification

Cavitation rate was determined by counting individual aggregates at 7 days of blastoid growth in at least 3 24-well wells per condition. Tile scan images of the entire well were stitched together and aggregates vs. cavitated aggregates were manually counted in ImageJ. Final means and standard deviations are reported in the graph among 2 biological replicates. Significance determined by a 1-way ANOVA with multiple comparisons. *p<0.05, **p<0.01.

Blastoid size was determined with ImageJ software by drawing a circle around the entire blastoid, and diameters determined by a single line drawn across the widest apparent part of the blastoid (day 7 n=1901; day 8 n=18). Inner cell mass size was determined by utilizing the OCT4-GFP channel and manually drawing around the brightest region of GFP+ areas (control n=152 blastoids; —CHIR n=130). Significance determined with Welch's t-test, ****p<0.0001.

scRNA-seq Analysis

Raw sequencing data were converted to fastq format using the cellranger mkfastq command (v.5.0.0). scRNA-seq reads were aligned to the GRCh38 (UCSC hg38) reference genome and quantified using the cellranger count command using default parameters.

Count data was processed using the R package Seurat (v.4.0.0) (Stuart et al., 2019), using Gencode v.31 for gene identification and considering only protein-coding genes. Cells with less than 2,500 or more than 8,000 informative genes expressed, cells with less than 15,000 sequenced fragments and cells with more than 25% mitochondrial gene content were excluded. Count data was log-normalized and scaled to 10,000. scRNA-seq data sets for H9 ESC- and CT30-derived TSC organoids, and replicates respectively, were transformed and mitochondrial gene content was regressed out using SCTransform as implemented in Seurat, and the data sets were integrated using 4,000 anchors following SC transformation.

PCA analysis was based on the 3,500 most variable genes. Nearest neighbors were computed using the top 6 principal components, and 5 clusters were identified using the Louvain community detection implemented in Seurat's FindClusters function (‘resolution=0.3’). 2-dimensional representations were generated using uniform manifold approximation and projection (UMAP). For each cluster, the genes differentially expressed between H9 ESC and CT30-derived organoids were determined using the FindMarkers function and a Wilcoxon Rank Sum test (‘logfc.threshold=0.1, min.pct=0.25’). GO term enrichment analyses were performed with the clusterProfiler package in R. To infer developmental trajectories, quality-filtered count data was normalized, and replicates were integrated using the ingest function in the Python package Scanpy (v.1.2.8; scanpy.readthedocs.io). Diffusion maps were calculated with destiny⁴⁶ as implemented in Scanpy.

To compare with publicly available scRNA-seq data sets, raw counts were obtained from GEO (accession: E-MTAB-3929²⁸; GSE136447²⁴), and re-analyzed as described above using SC transformation. For the Liu et al. data set, we used the markers provided as a Supplementary Table in the paper to reproduce and reassign the cell types to clusters; for the Xiang et al. data set, cell types were directly provided as Supplementary Table. Processed and annotated public scRNA-seq data sets were integrated with our combined TSC organoid scRNA-seq data sets using reciprocal PCA as implemented in Seurat.

What is claimed is:

1. A method for generating blastoids from naïve hPSCs, the method comprising:
exposing the naïve hPSCs to 5i/L/A;
aggregating the exposed hPSCs in N2B27; and
treating the aggregated hPSCs with a simplified blastocyst induction medium (“BIM”).

2. The method of claim 1, wherein:
the exposing is carried out for about 1 day;
the aggregating is carried out for about 2 days; and
the treating is carried out for about 5 days.

3. The method of claim 1, wherein the naïve hPSCs are WIBR3 OCT4-GFP naïve hPSCs.

4. The method of claim 1, wherein a GSK3a/P inhibitor is omitted.

5. The method of claim 4, wherein the omitted GSK3α/β inhibitor is CHIR99021.

6. The method of claim 1, wherein a BRAF inhibitor is omitted.

7. The method of claim 6, wherein the omitted BRAF inhibitor is SB590885.

8. The method of claim 1, wherein a leukemia inhibitory factor (LIF) is omitted.

9. The method of claim 1, wherein:
an Activin/Nodal/TGFβ inhibitor is omitted; and
an amount of A83-01 is increased.

10. The method of claim 9, wherein the omitted Activin/Nodal/TGFβ inhibitor is SB431542.

11. The method of claim 1, wherein Activin and FGF2 are omitted.

12. The method of claim 11, wherein the Activin and the FGF2 are omitted from the aggregating step.

13. The method of claim 1, wherein an amount of sodium pyruvate is increased.

14. The method of claim 1, wherein a seeding density is increased.

15. A system for extended culture of human blastoids from naïve hPSCs, the system comprising:
blastoids generated from naïve hPSCs by a method comprising:
exposing the naïve hPSCs to 5i/L/A;
aggregating the exposed hPSCs in N2B27; and
treating the aggregated hPSCs with a simplified blastocyst induction medium (“BIM”); and
a 3D matrix.

16. The system of claim 15, wherein:
the exposing is carried out for about 1 day;
the aggregating is carried out for about 2 days; and
the treating is carried out for about 5 days.

17. The system of claim 15, wherein the naïve hPSCs are WIBR3 OCT4-GFP naïve hPSCs.

18. The system of claim 15, wherein the 3D matrix comprises Cultrex.

19. The system of claim 15, wherein the 3D matrix comprises Matrigel.

20. The system of claim 15, wherein the 3D matrix comprises a combination of Cultrex and Matrigel.
